(19) United States
(12) Patent Application Publication
(10) Pub. No.: US 2018/0252700 A1
(43) Pub. Date: Sep. 6, 2018

(54) SCREENING AGENTS IN DRY EYE DISEASE

(71) Applicant: Mark B. Abelson, Teton Village, WY (US)

(72) Inventor: Mark B. Abelson, Teton Village, WY (US)

(73) Assignee: Ora, Inc., Andover, MA (US)

(21) Appl. No.: 15/726,690

(22) Filed: Oct. 6, 2017

Related U.S. Application Data
(60) Provisional application No. 62/450,214, filed on Jan. 25, 2017.
(60) Provisional application No. 62/404,902, filed on Oct. 6, 2016.

(51) Int. Cl. G01N 33/50; A61K 31/05; A61P 27/02

(52) U.S. Cl. CPC G01N 33/5008; G01N 2800/52; A61P 27/02; A61K 31/05

(57) ABSTRACT

The present invention provides methods of identifying a subject as susceptible to treatment for an ocular disease or disorder. The present invention also provides methods of screening an agent for the treatment of an ocular disease or disorder. In certain embodiments, the ocular disease or disorder is dry eye disease.

### RELATED APPLICATIONS

This application claims priority to U.S. Provisional Application No. 62/404,902 filed on Oct. 6, 2016, and U.S. Provisional Application No. 62/450,214, filed on Jan. 25, 2017, the entire contents of each of which are incorporated by reference in their entireties herein.

### BACKGROUND

Dry eye disease is a relatively common condition characterized by inadequate tear film protection of the cornea. Dry eye symptoms are most typically managed by artificial tears, which are often time consuming, frustrating, and frequently ineffective or variably effective treatments. Tens of millions of people are affected worldwide by dry eye, and nearly five million Americans 50 years of age and older are estimated to have dry eye. Of these, more than three million are women and more than one and a half million are men. Elderly people frequently experience dryness of the eyes, but dry eye can occur at any age. Dry eye is a potentially disabling disease adversely impacting the vision-related quality of life.

Current therapeutic options for dry eye disease are limited and costly. There remains a need in the art for new and improved screening tools to identify Dry Eye patients for treatment. Further, there remains a need in the art for new and improved methods of determining the efficacy of TRPM8 antagonists for treating dry eye disease.

### SUMMARY OF THE INVENTION

The present invention provides methods of identifying a subject as susceptible to treatment for an ocular disease or disorder. The present invention also provides methods of screening an agent for the treatment of an ocular disease or disorder.

In a first aspect, the present invention provides a method of identifying a subject as susceptible to treatment for an ocular disease or disorder comprising administering a TRPM8 agonist to the eye of the subject, detecting the effect of the TRPM8 agonist on the symptoms of the ocular disease or disorder, and identifying the subject as susceptible to treatment for the ocular disease or disorder based on the results of the detecting step.

In another aspect, the present invention provides a method of measuring the severity of the symptoms of an ocular disease or disorder in a subject, comprising administering a transient receptor potential melastatin 8 (TRPM8) agonist to the eye of the subject;

detecting the effect of the TRPM8 agonist on the symptoms of the ocular disease or disorder; and assessing the effect of the TRPM8 agonist on the severity of the symptoms of the ocular disease or disorder using one or more dry eye questionnaires.

In one embodiment, the one or more dry eye questionnaires is selected from the group consisting of: the 0-10 Cooling Scale, the 4-Symptom Questionnaire, the Ocular Surface Disease Index (OSDI, Allergan), the Symptom Assessment iN Dry Eye (SANDE), the Standard Patient Evaluation of Eye Dryness (SPEED, TearScience), the Dry Eye Questionnaire (DEQ, TearLab), the McMonnies Questionnaire, the Subjective Evaluation of Symptom of Dryness (SESoD, Allergan), the Impact of Dry Eye on Everyday Life (IDEEL, Alcon) and the Dry Eye-Related Quality-of-Life Score Questionnaire (DEQS, Dry Eye Society), or a combination thereof.

In one embodiment, the method further comprises treating the subject for the ocular disease or disorder.

In one embodiment, the TRPM8 agonist is menthol. In a further embodiment, the menthol is administered at a concentration of about 0.0001% to about 0.1% w/v. In another further embodiment, the menthol is administered at a concentration of about 0.000050% to about 0.000075% w/v.

In one embodiment, the effect of the TRPM8 agonist on the symptoms of the ocular disease or disorder is determined using one or more dry eye questionnaires. In a further embodiment, the one or more dry eye questionnaires is selected from the group consisting of: the 0-10 Cooling Scale, the 4-Symptom Questionnaire, the Ocular Surface Disease Index (OSDI, Allergan), the Symptom Assessment iN Dry Eye (SANDE), the Standard Patient Evaluation of Eye Dryness (SPEED, TearScience), the Dry Eye Questionnaire (DEQ, TearLab), the McMonnies Questionnaire, the Subjective Evaluation of Symptom of Dryness (SESoD, Allergan), the Impact of Dry Eye on Everyday Life (IDEEL, Alcon) and the Dry Eye-Related Quality-of-Life Score Questionnaire (DEQS, Dry Eye Society), or combinations thereof. In one embodiment, the effect of the TRPM8 agonist on the symptoms of the ocular disease or disorder is determined using a 0-10 Cooling Scale, 4-Symptom Questionnaire, the Ocular surface Disease Index, or a combination thereof. In preferred embodiments, the effect of the TRPM8 agonist on the symptoms of the ocular disease or disorder is determined using a 0-10 Cooling Scale. In a further embodiment, the method further comprises measuring the duration of the effect of the TRPM8 agonist on the symptoms of the ocular disease or disorder. In another further embodiment, the duration of the effect of the TRPM8 agonist on the symptoms of the ocular disease or disorder is used to identify the subject as susceptible to treatment for the ocular disease or disorder. In another further embodiment, a longer duration of symptoms identifies the subject as susceptible to treatment for the ocular disease or disorder. In one embodiment of any of the above aspects, the subject has a dry eye disease or disorder. In one embodiment, the subject has had the ocular disease or disorder for less than 10 years. In another embodiment, the subject has had the ocular disease or disorder for greater than 10 years.

In one embodiment, the subject identified as susceptible to treatment for the ocular disease or disorder is selected for a clinical trial.

In another aspect, the present invention features a method of screening an agent for the treatment of an ocular disease or disorder, comprising administering a TRPM8 agonist to the eye of a subject having an ocular disease or disorder, administering the agent to the eye of the subject at a concentration that alleviates symptoms of the ocular disease or disorder, and determining an effect of the agent on the symptoms of the ocular disease or disorder.

In another aspect, the present invention features a method of screening an agent for the treatment of an ocular disease or disorder, comprising administering the agent to the eye of a subject; administering a TRPM8 agonist to the eye of the subject to elicit symptoms of the ocular disease or disorder; and determining an effect of the TRPM8 agonist on the symptoms of the ocular disease or disorder in the subject. In one embodiment, the subject has an ocular disease or disorder. In another embodiment, an absence of symptoms of the ocular disease or disorder after administering the TRPM8 agonist indicates the agent is useful for the treatment of an ocular disease or disorder.

In one embodiment of any of the above aspects, the agent is a TRPM8 antagonist.

In one embodiment of any of the above aspects, the TRPM8 agonist is menthol. In another embodiment, the menthol is administered at a concentration of about 0.0001% to about 0.01% w/v. In one embodiment, the symptoms are determined using a 0-10 Cooling Scale, 4-Symptom Questionnaire, the Ocular surface Disease Index, or a combination thereof. In exemplary embodiments, the 0-10 Cooling Scale is used. In one embodiment of any of the above aspects, the effect of the agent on treating the ocular disease or disorder is an improvement of the symptoms of the ocular disease or disorder. In another embodiment of any of the above aspects, an improvement of the symptoms of the ocular disease or disorder indicates that the agent is useful for the treatment of an ocular disease or disorder.

In one embodiment of any of the above aspects, the ocular disease or disorder is ocular discomfort. In one embodiment of any of the above aspects, the ocular disease or disorder is dry eye disease or dry eye discomfort. In a further embodiment of any of the above aspects, the ocular disease or disorder is attributable to one or more causes selected from aging, contact lens usage, environmental fatigue, diet, hydration, systemic disease, inflammation or medication usage. In one embodiment of any of the above aspects, the ocular disease or disorder is due to excessively fast tear evaporation (evaporative dry eyes) or inadequate tear production. In one embodiment of any of the above aspects, the ocular disease or disorder is associated with refractive surgery.

In one embodiment of any of the above aspects, the TRPM8 antagonist is selected from the group consisting of: a small molecule, a nucleic acid molecule, an aptamer, an antisense molecule, an RNAi molecule, a protein, a peptide and an antibody or antibody fragment.

### BRIEF DESCRIPTION OF THE DRAWINGS

FIG. 1 is a graph that shows frequency distribution of symptom scores. FIG. 1 shows a non-normal distribution, skewed left.

FIG. 2 is a graph that shows FIG. 2: menthol symptom response (also referred to as “Cooling Response” (total symptom score—TSS) in Normal and Dry Eye patients over the range of concentrations from 0.00001% to 0.001% (Rohto (0.01% w/v) data is from a previous study, used in this experiment for visualized comparison). Stars represent significant differences between groups at a 95% confidence interval. Rohto refers to Menthol.

FIG. 3 is a graph that shows the “Upon Instillation” menthol symptom response in Normal and Dry Eye populations. No significant differences were found at any concentrations tested.

FIG. 4 is a graph that shows frequency distribution of the duration of symptom response post-ROHTO Hydra, in Dry Eye and Normal populations. Duration is defined as the furthest time point post-dose which elicited a symptom response of greater than 0 on the 0-10 Cooling Scale.

FIG. 5 is a graph that shows the average symptom response of Dry Eye and Normal groups secondary to ROHTO Hydra. Each time point, with the exception of Immediate, was significantly higher in dry eye compared to normal populations.

FIG. 6 is a graph that shows symptom response to SYSTANE Ultra. Scores represent mean response within each population, averaged over both eyes.

FIG. 7 is a graph that shows symptom response to ROHTO Hydra. Scores represent mean response within each population, averaged over both eyes.

FIG. 8 is a graph that shows normalized menthol symptom response across populations. Normalized data is ROHTO Hydra symptom response minus SYSTANE Ultra symptom response, at all time points. Note: Y-axis scale is now compressed.

FIG. 9 is a graph that shows normalized menthol symptom response in all Dry Eye patients vs normals. Normalized data is ROHTO Hydra symptom response minus SYSTANE Ultra symptom response, at all time points.

FIG. 10 is a graph that shows Menthol Total Symptom Score, in Normal and Dry Eye populations. Differences were significant (p=0.0004), and standard deviation is represented as error bars.

FIG. 11 is a graph that shows corneal sensitivity, averaged across all patients in each population. Significant differences within-population from baseline are displayed with an asterisk.

FIG. 12 is a graph that shows mean (±SD) of cooling response scores (0-10 scale) to TRPM8-agonist ROHTO Hydra (15 μL, 0.01% Menthol) OU for each time point post-dose in DED vs. normal subjects. Comparisons between groups were calculated using the Student t-test (asterisks indicates statistical significance, p<0.05).

FIG. 13 is a graph that shows mean (±SD) of cooling response scores (0-10 scale) to SYSTANE Ultra (15 μL, 0.01% Menthol) OU for each time point post-dose in dry eye disease (DED) vs. normal subjects. Comparisons between groups were calculated using the Student t-test (statistical significance, p<0.05).

FIG. 14 is a graph that shows mean (±SD) of the sum cooling scores (0-10 scale, 6 time points, range 0 to 60) post ROHTO Hydra and SYSTANE Ultra administration. Comparisons between DED and normal groups were calculated using the Student t-test (asterisk indicate statistical significance, p<0.05.

FIG. 15 is a graph that shows mean cooling scores post-ROHTO Hydra application as a function of time in patients who reported having DED for less than 10-years (N=18) vs. those having DED greater than 10-years (N=15). Groups are age-matched.

FIG. 16 is a graph that shows the cooling response secondary to SYSTANE ultra instillation in dry eye patients.

FIG. 17 is a graph that shows the cooling response secondary to ROHTO hydra instillation in dry eye patients.

FIG. 18 is a graph that shows average cooling symptom response secondary to instillation of 15 μl OU ROHTO hydra in male and female Dry Eye Patients.

FIG. 19 is a graph that shows cooling scores in sub-populations of Dry Eye subjects based on slope of their cooling response to ROHTO Hydra.

FIG. 20 is a graph that shows the effect of single doses of either 0.1% TRPM8, 0.05% TRPM8, 0.01% TRPM8, or water on tear production on day 16. Baselines were obtained on Days 10, 11 and 14. Tear production was measured at time points: 15 mins, 2 hours, 7 hours, 24 hours and 48 hours post dose. N=20. Graphs show the mean ±SEM.

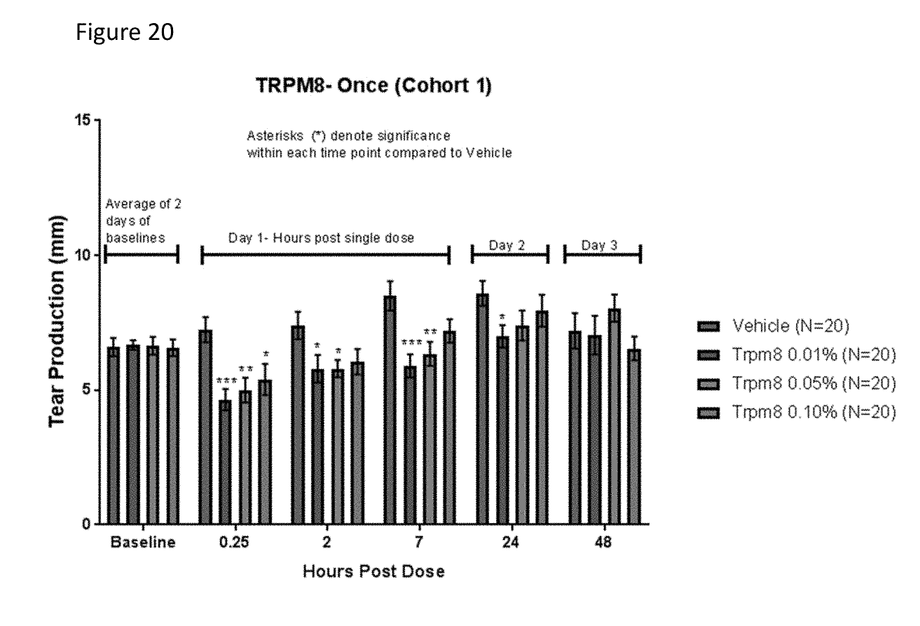

FIG. 21 is a graph that shows the effect of single doses of either 0.1% TRPM8, 0.05% TRPM8, 0.01% TRPM8, or water on tear production over three days (single dose given on days 9-11). Baselines were obtained on Days 1, 2 and 5. Tear production was measured at time points: 15 mins, 2 hours, 7 hours and 24 hours post 1st dose and last dose and 48 hours and 72 hours post last dose. N=20. Graphs show the mean ±SEM.

FIG. 22 is a graph that shows the effect of doses of either 0.1% TRPM8, 0.05% TRPM8, 0.01% TRPM8, or water twice on Days 43-44, once on Day 45. Baselines were obtained on Days 36, 29 and 40. Tear production was measured at time points: 15 mins, 2 hours, 7 hours and 24 hours post 1st dose and last dose and 48 hours post last dose. N=20. Graphs show the mean ±SEM.

FIG. 23 is a graph that shows the effect of single doses of either 0.1% TRPM8, 0.05% TRPM8, 0.01% TRPM8, or water, given immediately following menthol dose, on blink rate per minute of Balb/C mice 15 minutes and 24 hours post TRPM8 dose. N=10.

### DETAILED DESCRIPTION

The present invention is based, in part, on the surprising finding that dry eye populations can differentiate menthol from a placebo at concentrations lower than normal. The present invention reports the unexpected finding that the menthol concentration can be lowered considerably for a more precise diagnostic tool, and that duration of menthol-induced symptoms can be used as a separator of normal and dry eye groups.

### Definitions

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The terms “administer”, “administering” or “administration” include any method of delivery of a pharmaceutical composition or agent into a subject's system or to a particular region in or on a subject. In certain embodiment, the composition is administered topically to the eye. The eye comprises a tissue or gland in or around the eye selected from the group consisting of ocular tissue, eyelids of the subject, ocular surface, meibomian gland and or lacrimal gland. Administration topically to the eye is meant to include administration to the eye or the area around the eye. Administering an agent can be performed by a number of people working in concert. Administering an agent includes, for example, prescribing an agent to be administered to a subject and/or providing instructions, directly or through another, to take a specific agent, either by self-delivery, or for delivery by a trained professional.

A “therapeutically effective amount” is that amount sufficient to treat a disease in a subject. A therapeutically effective amount can be administered in one or more administrations.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease or condition, stability (i.e., not worsening) state of disease or condition, amelioration or palliation of the disease state, and prevention of the disease state. In certain embodiments, alleviating symptoms of an ocular disease or disorder, refers to lessening, diminishing or not worsening the signs or symptoms of the ocular disease or disorder. A subject who is “susceptible to treatment” is a subject that may be treated for an ocular disease or disorder.

Treatment does not need to be curative. Treatment can also refer to prevention of one or more signs or symptoms of an ocular disease or disorder. U.S. Provisional Application No. 62/237,672, filed on Oct. 6, 2015, incorporated by reference in its entirety herein, teaches methods of treating or preventing an ocular disease or disorder in a subject comprising administering to the subject a composition comprising a pharmaceutically acceptable carrier and a pharmaceutically effective amount of a transient receptor potential melastatin 8 (TRPM8) antagonist.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the term “ocular disease or disorder” is meant to refer to any disease or disorder of or associated with the eye, including symptoms of the ocular disease or disorder. In certain embodiments, the ocular disease or disorder is dry eye disease, including symptoms of dry eye disease.

The term “ocular pain or discomfort” is meant to include, but not be limited to, ache, dryness or itchiness, a gritty sensation, redness, sensitivity to light, stinging or burning sensation or pain associated with the eye.

As used herein, the term “dry eye disease” is meant to refer to an eye disease caused by decreased tear production or increased tear film evaporation. Other names for dry eye include dry eye syndrome, keratoconjunctivitis sicca (KCS), dysfunctional tear syndrome, lacrimal keratoconjunctivitis, evaporative tear deficiency, aqueous tear deficiency, and LASIK-induced neurotrophic epitheliopathy (LNE).

In certain embodiments, the dry eye disease or disorder is attributable to one or more causes selected from, but not limited to, aging, contact lens usage, environmental stress, fatigue, diet, hydration, systemic disease, visual tasking (such as reading or video screen use), inflammation or medication usage. In other embodiment, the dry eye disease is due to excessively fast tear evaporation (evaporative dry eyes) or inadequate tear production. In other embodiments, the dry eye disease is associated with refractive surgery.

As used herein, the “transient receptor potential melastatin 8 (TRPM8) agonist” is meant to refer to any compound or any agent that can activate the TRPM8 receptor. Examples of TRPM8 agonists include, but are not limited to, alinalool, geraniol, hydroxy-citronellal, WS-3, WS-23, Frescolat MGA, Frescolat ML, PMD 38, Coolact P, and Cooling Agent. In certain embodiments, the TRPM8 agonist is menthol.

As used herein, the term “transient receptor potential melastatin 8 (TRPM8) antagonist” is meant to refer to any compound or any agent that can inhibit the activity of TRPM8 (i.e. block TRPM8-mediated signaling cascade) at an ophthalmically relevant concentration. TRPM8 antagonists useful in the methods of the invention include, but are not limited to, a small molecule, a nucleic acid molecule, an aptamer, an antisense molecule, an RNAi molecule, a protein, a peptide and an antibody or antibody fragment. Exemplary TRPM8 antagonists are described in W02006040136, incorporated by reference in its entirety herein.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### I. Ocular Diseases or Disorders

The present invention demonstrates the unexpected finding that the TRPM8 agonist menthol can be used as a screening tool for dry eye studies.

Ocular diseases or disorders include any disease or disorder of or associated with the eye itself, or a tissue or gland in or around the eye, for example ocular tissue, eyelids of the subject, ocular surface, meibomian gland and or lacrimal gland.

An ocular disease or disorder that is treated or prevented by the methods of the present invention is dry eye disease. Other names for dry eye include dry eye syndrome, keratoconjunctivitis sicca (KCS), dysfunctional tear syndrome, lacrimal keratoconjunctivitis, evaporative tear deficiency, aqueous tear deficiency, and LASIK-induced neurotrophic epitheliopathy (LNE).

Dry eye disease is caused by an inadequate or altered tear film, and may be the result of an inability of the lacrimal glands to produce an adequate quantity of tears with the proper composition (Abelson et al. Curr Opin Ophthalmol 20: 282-286, 2009; Barabino and Dana Chem Immunol Allergy 92: 176-184, 2007). Alternatively, dry eyes may result from an inability of sensory afferent neurons to monitor the corneal surface, resulting in insufficient neuronal drive to produce a sufficient quantity of tears (Dartt Ocul Surf 2: 76-91, 2004, Prog Retin Eye Res 28: 155-177,2009; Mathers CLAO J 26: 159-165, 2000; van Bijsterveld et al. Br J Ophthalmol 87: 128-130, 2003). Corneal primary afferent neurons express a range of membrane channels, which corresponds to their physiological characteristics. The primary afferent neurons innervating the cornea regulate secretion of basal tearing with a relay through the spinal trigeminal nucleus. It has been proposed that polymodal nociceptors express channels responding to noxious chemical, thermal, and mechanical stimulation, including TRPV1, TRPA1, TRPV4, and acid sensing ion channels (ASIC) channels. In contrast, cold receptors express TRPM8 channels, which are sensitive to innocuous cooling (Meng and Kurose Experimental Eye Research 117 (2013) 79-87).

Furthermore, it has been proposed that even if the initial cause of dry eye is dysfunction of the lacrimal gland, it has been suggested that the dry eye condition itself may affect corneal afferents involved in tear regulation, initiating a vicious cycle that may lead to a further deterioration in lacrimal gland function and a worsening of the condition (Mathers, 2000).

Aqueous tear-deficient dry eye is a disorder in which the lacrimal glands fail to produce enough of the watery component of tears to maintain a healthy eye surface. Evaporative dry eye may result from inflammation of the meibomian glands, also located in the eyelids. These glands make the lipid or oily part of tears that slows evaporation and keeps the tears stable. Dry eye can be associated with inflammation of the surface of the eye, the lacrimal gland, or the conjunctiva; any disease process that alters the components of the tears; an increase in the surface of the eye, as in thyroid disease when the eye protrudes forward; cosmetic surgery, if the eyelids are opened too widely.

Symptoms of dry eye include, but are not limited to stinging or burning of the eye; a sandy or gritty feeling as if something is in the eye; episodes of excess tears following very dry eye periods; a stringy discharge from the eye; pain and redness of the eye; episodes of blurred vision; heavy eyelids; inability to cry when emotionally stressed; uncomfortable contact lenses; decreased tolerance of reading, working on the computer, or any activity that requires sustained visual attention; eye fatigue.

Dry eye can be a temporary or chronic condition. Severe dry eye is a debilitating disease that affects millions of patients worldwide and can cripple some patients. Millions of these individuals suffer from the most severe form. This disease often inflicts severe ocular discomfort, results in a dramatic shift in quality of life, induces poor ocular surface health, substantially reduces visual acuity and can threaten vision. Patients with severe dry eye develop a sensitivity to light and wind that prevents substantial time spent outdoors, and they often cannot read or drive because of the discomfort.

The following are non-limiting examples of causes and symptoms of ocular diseases or disorders, such as ocular discomfort, and dry eye or severe dry eye. Ocular discomfort and/or dry eye can be a side effect of some medications, including antihistamines, nasal decongestants, tranquilizers, certain blood pressure medicines, Parkinson's medications, birth control pills and anti-depressants. Environmental stress dry environments or with moving air is a big factor in causing ocular discomfort and dry eye. Aging is one of the most common causes of ocular discomfort and/or dry eyes. About half of all people who wear contact lenses complain of ocular discomfort and/or dry eyes. Skin disease on or around the eyelids can result in ocular discomfort and/or dry eye. Diseases of the glands in the eyelids, such as meibomian gland dysfunction, can cause ocular discomfort and/or dry eye. Ocular discomfort and/or dry eye can occur in women who are pregnant. Women who are on hormone replacement therapy may experience ocular discomfort and/or dry eye symptoms. Ocular discomfort and/or dry eye can also develop after the refractive surgery known as LASIK. Symptoms of dry eye associated with refractive surgery have been reported to last in some cases from six weeks to six months or more following surgery. Ocular discomfort and/or dry eye can result from chemical and thermal burns that scar the membrane lining the eyelids and covering the eye. Allergies can be associated with ocular discomfort and/or dry eye. Infrequent blinking, associated with staring at computer or video screens or other visual tasks like reading, may also lead to ocular discomfort and/or dry eye symptoms. Both excessive and insufficient dosages of vitamins can contribute to ocular discomfort and/or dry eye. Loss of sensation in the cornea from long-term contact lens wear can lead to ocular discomfort and/or dry eye. Ocular discomfort and/or dry eye can be associated with immune system disorders such as Sjögren's syndrome, lupus, and rheumatoid arthritis. Sjögren's leads to inflammation and dryness of the mouth, eyes, and other mucous membranes. It can also affect other organs, including the kidneys, lungs and blood vessels. Ocular discomfort and/or dry eye can be a symptom of chronic inflammation of the conjunctiva, the membrane lining the eyelid and covering the front part of the eye, or the lacrimal gland. Ocular discomfort and/or inflammation can be caused by certain eye diseases, infection, exposure to irritants such as chemical fumes and tobacco smoke, or drafts from air conditioning or heating. If the surface area of the eye is increased, as in thyroid disease when the eye protrudes forward or after cosmetic surgery if the eyelids are opened too widely, ocular discomfort and/or dry eye can result. Ocular discomfort and/or dry eye may occur from exposure keratitis, in which the eyelids do not close completely during sleep.

### II. TRPM8 Antagonists

Transient Receptor Potential (TRP) channels are one of the largest group of ion channels and, based on their sequence homology, are classified into 6 sub-families (TRPV, TRPM; TRPA, TRPC, TRPP and TRPML). TRP channels are cation-selective channels activated by several physical (such as temperature, osmolarity and mechanical stimuli) and chemical stimuli. TRPM8, which was cloned in 2002, is a non-selective cation channel of the TRP family TRPM8 is located on primary nociceptive neurons (A-delta and C-fibers) and is also modulated by inflammation-mediated second messenger signals (Abe, J., et al., Neurosci Lett 2006, 397(1-2), 140-144; Premkumar, L. S., et al., J. Neurosci, 2005, 25(49), 11322-1 1329). It is activated by mild cold temperatures and synthetic cool-mimetic compounds such as menthol, eucalyptol and icilin (McKemy D. D. et al., Nature (2002) 416, 52-58; Peier A. M. et al. Cell (2002) 108, 705-715). Like several other TRP channels, TRPM8 is also gated by voltage (Nilius B. et al., J. Physiol. (2005) 567, 35-44). The voltage dependence of TRPM8 is characterized by a strong outward rectification at depolarized transmembrane potential and a rapid and potential-dependent closure at negative membrane potentials. Cooling agents and menthol application shifts the activation curve towards more negative potentials, increasing the possibility for the opening of the channel and boosting inward currents at physiological membrane potentials. Other endogenous factors, such as phospholipase A2 products (Vanden Abeele F. et al., J. Biol. Chem. (2006) 281, 40174-40182), endocannabinoids (De Petrocellis Let al., Exp. Cell. Res. (2007) 313, 1911-1920) and PIP2 (Rohacs T. et al., Nat. Neurosci. (2005) 8, 626-634) also participate in channel regulation.

TRPM8 antagonists can include, but are not limited to small molecules, nucleic acid molecules, aptamers, antisense molecules, RNAi molecules, proteins, peptides and antibodies or antibody fragments.

### Small Molecule Inhibitors

Several classes of non-peptide TRPM8 antagonists have been disclosed. International patent application WO 2006/040136 describes substituted 4-benzyloxy-phenylmethylamide derivatives as cold menthol receptor-1 (CMR-I) antagonists for the treatment of urological disorders. International patent applications WO 2007/0 17092A1, WO 2007/017093A1 and WO 2007/0 17094A1 describe benzyloxyphenylmethyl carbamate, substituted 2-benzyloxybenzoic acid amide and substituted 4-benzyloxybenzoic acid amide derivatives for the treatment of diseases associated with the cold menthol receptor (CMR), a.k.a. TRPM8; WO 2007/134107 describes phosphorous-bearing compounds as TRPM8 antagonists for the treatment of TRPM8-related disorders; WO 2009/012430 describes sulfonamides for the treatment of diseases associated with TRPM8; WO 2010/103381 describes the use of spirocyclic piperidine derivatives as TRPM8 modulators in prevention or treatment of TRPM8-related disorders or diseases; and, WO 2010/125831 describes sulfamoyl benzoic acid derivatives as modulators of the TRPM8 receptor and their use in the treatment of inflammatory, pain and urological disorders.

Other TRPM8 inhibitors include AMTB (N-(3-aminopropyl)-2-[(3-methylphenyl)me thoxy]-N-(2-thienylmethyl)-benzamidehydrochloride (1:1) hyclate) (CAS 926023-82-7) (Santa Cruz Biotechnology, sc-361103) and JNJ41876666 (compound 5 in Parks D. J. et al., 2011, J Med Chem 54: 233-247); 3-[7-Trifluoromethyl-5-(2-trifluoromethyl-phenyl)-1H-benzimidazol-2-yl]-1-oxa-2-aza-spiro[4.5]dec-2-eneHydrochloride,). BCTC, thio-BCTC, and capsazepine were identified as antagonists of the TRPM8 receptor. These antagonists physically block the receptor for cold and menthol, by binding to the S1-S4 voltage-sensing domain, preventing response (Behrendt H. J. et al., Br. J. Pharmacol. 141 (4): 737-45).

In certain preferred embodiments of the invention, the TRPM8 antagonist is a small molecule inhibitor.

Parks D. J. et al., J. Med. Chem. 2011, 54, 233-247 describe the design, synthesis, and optimization of a class of selective TRPM8 antagonists based on a benzimidazole scaffold.

In exemplary embodiments, the TRPM8 antagonist is the commercially available TRPM8 antagonist N-(2-Aminoethyl)-N-(4-(benzyloxy)-3-methoxybenzyl)thiophene-2-carboxamide hydrochloride, N-(2-Aminoethyl)-N-[[3-methoxy-4-(phenylmethoxy)phenyl]methyl]-2-thiophenecarboxamide hydrochloride (Sigma Aldrich M8-B hydrochloride (SML0893)), shown below as Compound I

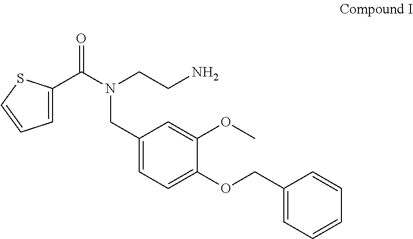 [SMILES: COc1cc(CN(CCN)C(=O)c2cccs2)ccc1OCc1ccccc1]

### Nucleic Acid Inhibitors

Antisense Molecules

The TRPM8 antagonist can be an antisense molecule that reduces transcription and/or translation of a component of TRPM8 activity. The antisense molecule comprises RNA or DNA prepared using antisense technology, where, for example, an antisense RNA or DNA molecule acts to block directly the translation of mRNA by hybridizing to targeted mRNA and preventing protein translation. Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such oligonucleotides can also be delivered to cells such that the antisense RNA or DNA may be expressed in vivo to inhibit production of components TRPM8 activity.

TRPM8 antagonists include antisense or sense oligonucleotides comprising a single-stranded nucleic acid sequence (either RNA or DNA) capable of binding to target mRNA (sense) or DNA (antisense) sequences. Such a fragment generally comprises about 10 to 40 nucleotides in length, preferably at least about 14 nucleotides, preferably from about 14 to 30 nucleotides.

Antisense or sense oligonucleotides further comprise oligonucleotides having modified sugar-phosphodiester backbones that are resistant to endogenous nucleases, or are covalently linked to other moieties that increases affinity of the oligonucleotide for a target nucleic acid sequence, or intercalating agents to modify binding specificities of the antisense or sense oligonucleotide for the target nucleotide sequence.

Small Interfering RNA (siRNA)

siRNA can be used as a TRPM8 antagonist, for example to inhibit TRPM8 activity. “siRNA” or “RNAi” are double-stranded RNA molecules, typically about 21 nucleotides in length, that are homologous to a gene or polynucleotide that encodes the target gene and interfere with the target gene's expression.

Nucleic Acid Molecules in Triple-Helix Formation

Nucleic acid molecules in triple-helix formation can be used as a TRPM8 antagonist. Nucleic acid molecules in triple-helix formation used to inhibit transcription should be single-stranded and composed of deoxynucleotides. A DNA oligonucleotide is designed to be complementary to a region of the gene involved in transcription. The base composition of these oligonucleotides is designed such that it promotes triple-helix formation via Hoogsteen base-pairing rules, which generally require sizeable stretches of purines or pyrimidines on one strand of a duplex.

Ribozymes

Ribozymes can be used as a TRPM8 antagonist. A “ribozyme” is an enzymatic RNA molecule capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques.

### Antibodies

The term “antibody” is used in the broadest sense and specifically covers, for example, polyclonal antibodies, monoclonal antibodies (including antagonist and neutralizing antibodies), antibody compositions with polyepitopic specificity, single chain antibodies, and fragments of antibodies, provided that they exhibit the desired activity of a TRPM8 inhibitor. Antagonistic TRPM8 antibodies are useful in the methods of the invention. An antibody inhibitor will specifically bind to a particular polypeptide or epitope on a particular polypeptide without substantially binding to any other polypeptide or polypeptide epitope. Such binding will partially or fully block, neutralize, reduce or antagonize TRPM8 activity.

An “isolated antibody” is one which has been identified and separated and/or recovered from a component of its natural environment. Contaminant components of its natural environment are materials which would interfere with diagnostic or therapeutic uses for the antibody, and may include enzymes, hormones, and other proteinaceous or non-proteinaceous solutes. Generally, the antibody will be purified (1) to greater than 95% by weight of antibody as determined by the Lowry method, and most preferably more than 99% by weight, (2) to a degree sufficient to obtain at least 15 residues of N terminal or internal amino acid sequence by use of a spinning cup sequenator, or (3) to homogeneity by SDS PAGE under reducing or non-reducing conditions using Coomassie blue or, preferably, silver stain. An isolated antibody includes the antibody in situ within recombinant cells since at least one component of the antibody's natural environment will not be present. Ordinarily, however, isolated antibody will be prepared by at least one purification step.

Where antibody fragments are used, the smallest inhibitory fragment that specifically binds to the target TRPM8 protein is preferred.

Various inhibitory TRPM8 antibodies are known in the art and are commercially available, for example from Santa Cruz Biotechnology TRPM8 (G-16), TRPM8 (N-15) or TRPM8 (D-25).

The term antibody is meant to include polyclonal antibodies, monoclonal antibodies, humanized antibodies, chimeric antibodies, antibody fragments, single chain antibodies, diabodies, bispecific antibodies and multivalent antibodies.

### III. Methods

The present invention demonstrates the unexpected finding that the TRPM8 agonist menthol can be used as a screening tool for dry eye studies. The present invention is based on the surprising finding that administration of a small amount of a TRPM8 agonist (e.g., menthol) alleviates discomfort in subjects that fit the inclusion criteria, but not in the subjects that do not fit. Accordingly, the present invention allows the screening of more patients more rapidly so it increases the capacity to enrich the patient population. Further, the present invention will lead to a considerable savings of time and money in clinical trials.

In a first aspect, the present invention provides a method of identifying a subject as susceptible to treatment for an ocular disease or disorder comprising administering a TRPM8 agonist to the eye of the subject, detecting the effect of the TRPM8 agonist on the symptoms of the ocular disease or disorder, and identifying the subject as susceptible to treatment for the ocular disease or disorder based on the results of the detecting step.

In one embodiment, the method further comprises treating the subject for the ocular disease or disorder.

In another aspect, the present invention provides a method of measuring the severity of the symptoms of an ocular disease or disorder in a subject, comprising administering a transient receptor potential melastatin 8 (TRPM8) agonist to the eye of the subject; detecting the effect of the TRPM8 agonist on the symptoms of the ocular disease or disorder; and assessing the effect of the TRPM8 agonist on the severity of the symptoms of the ocular disease or disorder using one or more dry eye questionnaires.

In one embodiment, the TRPM8 agonist is menthol. In one embodiment, the menthol is preferably administered at a concentration of about 0.00001% to about 0.1% w/v. In a further embodiment, the menthol is preferably administered at a concentration of about 0.0001 to about 0.01 w/v. In another further embodiment, the menthol is administered at a concentration of about 0.000050% to about 0.000075% w/v.

In one embodiment, the effect of the TRPM8 agonist on the symptoms of the ocular disease or disorder is determined using a 0-10 Cooling Scale, 4-Symptom Questionnaire, the Ocular surface Disease Index, or a combination thereof. In preferred embodiments, the 0-10 Cooling Scale is used.

The 0-10 Cooling Scale is an 11-point (0 to 10) scale, where 0=not cool and 10=very cool, and is described herein in the Examples.

Dry eye questionnaires known in the art include the Ocular Surface Disease Index (OSDI, Allergan), Symptom Assessment iN Dry Eye (SANDE), Standard Patient Evaluation of Eye Dryness (SPEED, TearScience), Dry Eye Questionnaire (DEQ, TearLab), McMonnies Questionnaire, Subjective Evaluation of Symptom of Dryness (SESoD, Allergan), Impact of Dry Eye on Everyday Life (IDEEL, Alcon) and Dry Eye-Related Quality-of-Life Score Questionnaire (DEQS, Dry Eye Society).

The Ocular Surface Disease Index (OSDI), developed by the Outcomes Research Group at Allergan Inc (Irvine, Calif.), (Walt J G Rowe M M Stern K L Evaluating the functional impact of dry eye: the Ocular Surface Disease Index. Drug Inf J. 1997; 311436) is a 12-item questionnaire designed to provide a rapid assessment of the symptoms of ocular irritation consistent with dry eye disease and their impact on vision-related functioning. The initial OSDI items were generated from patient comments from several years of clinical studies, several quality-of-life instruments, and suggestions from clinical investigators. This item list was then distributed to more than 400 patients with dry eye disease, who were asked to indicate whether they experienced any of the symptoms or problems on the list and, if so, how often. This information was combined with responses from 44 patients with dry eye disease and 2 health professionals who were asked to list aspects of their dry eye condition that affected their daily activities. Item responses were then categorized, and categories mentioned more than once were formatted into an initial questionnaire. This initial questionnaire included 40 items, which were later reduced to the final 12 questions on the basis of validity and reliability data from 3 groups (2 small groups of patients with dry eye and one phase II clinical trial group).

Symptom Assessment iN Dry Eye (SANDE) is a 2-item frequency- and severity-based visual analog scale that may also be used to assess symptoms in dry eye (Shaumberg et al. Ocul Surf 5: 50-57. doi:10.1016/51542-0124(12)70053-8). The SANDE is a simple dry eye instrument containing two items measuring the frequency and severity of symptoms, each was assessed on a 100 mm visual analog scale (VAS) ranging from ‘Rarely/Very mild’ to ‘All the time/Very severe’ and scored from 0 to 100, from which a SANDE Total score (also ranging from 0 to 100) is calculated as the square-root of the product of the two item scores.

The SPEED questionnaire assesses frequency and severity of patients' dry eye symptoms. It examines the occurrence of symptoms on the current day, past 72 hours and past three months (Ngo et al., Cornea. 2013 September; 32(9):1204-10). The SPEED questionnaire consists of four questions, and the SPEED score is then tallied to quickly obtain relevant dry eye patient symptom information.

The 4 Symptom Questionnaire is on a scale of 0-4, where 0=No Discomfort 1=Intermittent Awareness 2=Constant Awareness 3=Intermittend Discomfort 4=Constant Discomfort.

In a further embodiment, the method further comprises measuring the duration of the effect of the TRPM8 agonist on the symptoms of the ocular disease or disorder. In another further embodiment, the duration of the effect of the TRPM8 agonist on the symptoms of the ocular disease or disorder is used to identify the subject as susceptible to treatment for the ocular disease or disorder. In one embodiment, the duration is between 1-10 minutes, between 1-5 minutes, preferably between 1-4 minutes. In certain embodiments, a longer duration of symptoms may identify the subject as susceptible to treatment for the ocular disease or disorder. In exemplary embodiments, it has been found that the pattern of symptom progression is distinct between dry eye and normal populations. In certain embodiments, clear separation may be found at 1, 2, 3 or 4 minutes after treatment with the TRPM8 agonist.

In certain embodiments, the subject has had the ocular disease or disorder for less than 10 years. In another embodiment, the subject has had the ocular disease or disorder for greater than 10 years. Accordingly, certain sub-populations of subjects can be identified as being susceptible to treatment for the ocular disease or disorder.

In one embodiment, the subject identified as susceptible to treatment for the ocular disease or disorder is selected for a clinical trial.

In another aspect, the present invention features a method of screening an agent for the treatment of an ocular disease or disorder, comprising administering a TRPM8 agonist to the eye of a subject having an ocular disease or disorder, administering the agent to the eye of the subject at a concentration that alleviates symptoms of the ocular disease or disorder, and determining an effect of the agent on the symptoms of the ocular disease or disorder.

It is also contemplated by the present invention that the agent is administered prior to the TRPM8 agonist. Accordingly, in another aspect, the present invention features a method of screening an agent for the treatment of an ocular disease or disorder, comprising administering the agent to the eye of a subject having an ocular disease or disorder at a concentration that alleviates symptoms of the ocular disease or disorder, and then administering a TRPM8 agonist to the eye of the subject, and determining an effect of the agent on the symptoms of the ocular disease or disorder.

In another aspect, the present invention features a method of screening an agent for the treatment of an ocular disease or disorder, comprising administering the agent to the eye of a subject; administering a TRPM8 agonist to the eye of the subject to elicit symptoms of the ocular disease or disorder; and determining an effect of the TRPM8 agonist on the symptoms of the ocular disease or disorder in the subject. In one embodiment, the subject has an ocular disease or disorder. In another embodiment, an absence of symptoms of the ocular disease or disorder after administering the TRPM8 agonist indicates the agent is useful for the treatment of an ocular disease or disorder. For example, an absence of discomfort when the agonist is given would indicate that the agent worked.

In one embodiment, the agent is a TRPM8 antagonist.

In one embodiment, the TRPM8 agonist is menthol. In another embodiment, the menthol is administered at a concentration of about 0.0001% to about 0.01% w/v. In one embodiment, the symptoms are determined using a 0-10 Cooling Scale, 4-Symptom Questionnaire, the Ocular surface Disease Index, or a combination thereof. In preferred embodiments, the 0-10 Cooling scale is used. In one embodiment, the effect of the agent on treating the ocular disease or disorder is an improvement of the symptoms of the ocular disease or disorder. In another embodiment, an improvement of the symptoms of the ocular disease or disorder indicates that the agent is useful for the treatment of an ocular disease or disorder.

In one embodiment of any of the above aspects, the ocular disease or disorder is ocular discomfort. In one embodiment of any of the above aspects, the ocular disease or disorder is dry eye disease or dry eye discomfort. In a further embodiment of any of the above aspects, the ocular disease or disorder is attributable to one or more causes selected from aging, contact lens usage, environmental fatigue, diet, hydration, systemic disease, inflammation or medication usage. In one embodiment of any of the above aspects, the ocular disease or disorder is due to excessively fast tear evaporation (evaporative dry eyes) or inadequate tear production. In one embodiment of any of the above aspects, the ocular disease or disorder is associated with refractive surgery.

### V. Animal Models

A number of animal models of that mimic the different pathophysiologic mechanisms of dry eye have been described (Schrader S. et al., Dev Ophthalmol 2008, 41:298-312). Some of those models include the hereditary mouse models resembling Sjögren's syndrome (Schenke-Layland K. et al., Exp Eye Res 2010, 90(2):223-237; Lavoie T. N. et al., J Biomed Biotechnol 2011, 2011:549107), the mouse model induced by botulinum toxin B (Suwan-apichon 0. et al., Invest Ophthalmol Vis Sci 2006, 47(1):133-139) or controlled environment (Chen W. et al., Invest Ophthalmol Vis Sci 2008, 49(4):1386-1391), rat models induced by evoked dacryoadenitis (Jiang G. Invest Ophthalmol Vis Sci 2009, 50(5):2245-2254) or anticholinergic drugs (Jain P. et al., Exp Eye Res 2011, 93(4):503-512), rabbit models induced by closure of the meibomian gland orifices (Gilbard J. P. et al., Ophthalmology 1989, 96(8):1180-1186), controlled environment (Fujihara T. et al., J Ocul Pharmacol Ther 1995, 11(4):503-508), evoked dacryoadenitis (Guo Z. et al., Exp Eye Res 2000, 71(1):23-31), preganglionic parasympathetic denervation (Toshida H. et al., Invest Ophthalmol Vis Sci 2007, 48(10):4468-4475), topical medication of a preservative (Xiong C. et al., Invest Ophthalmol Vis Sci 2008, 49(5):1850-1856) or removing of the lacrimal gland (Chen Z. Y. et al., Cornea 2011, 30(9):1024-1029), canine models formed spontaneously (Hick S. J. et al., Exp Eye Res 1998, 67(6):709-718) or induced by canine distemper virus (de Almeida D. E. et al., Vet Ophthalmol 2009, 12(4):211-215) and monkey models by removing the lacrimal gland (Francois J. et al., Ophthalmic Res 1976, 8:414-424).

Studies in rats have used lacrimal gland removal to produce a dry condition on the ocular surface (Fujihara T. et al., Invest Ophthalmol Vis Sci 42: 96-100, 2001; Kaminer et al., J Neurosci 31: 11256-11267, 2011).

Because rabbits have large eyes amenable to slit-lamp microscopic examinations, and considering their gentle nature and relatively low cost to maintain, rabbit models are well-suited to study the development of dry eye. In one rabbit model, the lacrimal gland is disabled and the Harderian gland and nicitating membrane are surgically removed simultaneously (Francois J. et al., 1976; Gilbard J. P. et al., Invest Ophthalmol Vis Sci 1987, 28(2):225-228; Gilbard J. P. Acta Ophthalmol Suppl 1989, 192:95-101). Another study (Xie H. P. et al., Chin Ophthalmic Res 1992, 10(1):10-12) established a dry eye model in rabbits by burning the bulbar conjunctiva with 50% trichloroacetic acid then surgically removing the lacrimal gland, Harderian gland, and nictitating membrane.

Rahman W. et al., Pain 156 (2015) 942-950 describe exorbital gland removal, a model for aqueous tear deficient dry eye, to determine the effects of persistent reduced tear volume on the properties of ocular-responsive neurons at the Vi/Vc transition and Vc/C1 regions and evoked eye blink behavior.

Depending on the specific animal model selected and the time of intervention, e.g., before or after the appearance of metabolic syndrome, the animal models can be used to demonstrate the efficacy of the methods provide herein.

### EXAMPLES

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1

### Duration-Dependent Symptom Response to Menthol Separates Normal From Dry Eye Populations

### Methods

The following experiments were performed using, but not limited to, the methods descried below.

6 Healthy and 4 Dry Eye individuals participated in a 2-day internal pilot study. Each participant underwent randomized eye, contralateral, 15 μl pipette-instilled dosing of menthol and placebo (Sterile PBS, pH 7.2), across an escalating range of Menthol concentrations. The following dilutions (USP-grade L-Menthol in Sterile PBS, pH 7.2) were initially tested, in the following order (concentrations are w/v):

0.000001%

0.00001%

0.0001%

0.001%.

These concentrations represent 1/10000, 1/1000, 1/100, and 1/10 dilutions relative to ROHTO Hydra. Upon data analysis from Visit 1 (see below), a second visit was implemented and the following concentrations were tested (spanning the 1/1000 to 1/100 range of ROHTO Hydra):

0.000025%

0.000050%

0.000075%

Post dose, a 0-10 Cooling Scale questionnaire, where 0 represents “No cooling sensation”, and 10 denotes “Maximum cooling sensation”, was asked at the following time points (OD, OS separate; OD represents your right eye and OS represents your left eye): Upon instillation, 30 seconds, 1 minute, 2 minutes, 3 minutes, and 4 minutes—post dose. At least 45 minutes was allowed between each successive dosing to allow for re-sensitization of TRPM8, as well as to allow any analgesic effects of μ-opioid activity to diminish. At the beginning of Visit 1, symptom questionnaires and Ocular Surface Disease Index (OSDI) were asked. After all concentrations of menthol had been tested, Fluorescein staining and Anesthetized Schirmer's test were performed (1 hour washout). The Schirmer test, first described in 1903 by Schirmer, is still the method most commonly used to evaluate aqueous tear production (Schirmer Graefes Arch Clin Exp Ophthalmol. 1903; 56:197-291,1903).

Population Comparison

The dry eye and normal populations examined in this study were taken from in-office participants; however several indicators demonstrate that these populations are close to true normals and dry eye. Baseline symptom scores, OSDI, Fluorescein Staining, and Schirmer's are shown in Table 1, below.

TABLE 1
Parameter comparisons between populations.

| | Normal | Dry Eye | p-value |
|---|---|---|---|
| 4-Symptom | 0.66 | 11 | 0.01 |
| Questionaire | | | |
| OSDI | 3.5 | 6.8 | 0.05 |
| Fluorescein Staining | 1.66 | 5.95 | 0.03 |
| Anesthetized | 13.25 | 8.4 | 0.11 |
| Schirmers | | | |

Statistical Analysis

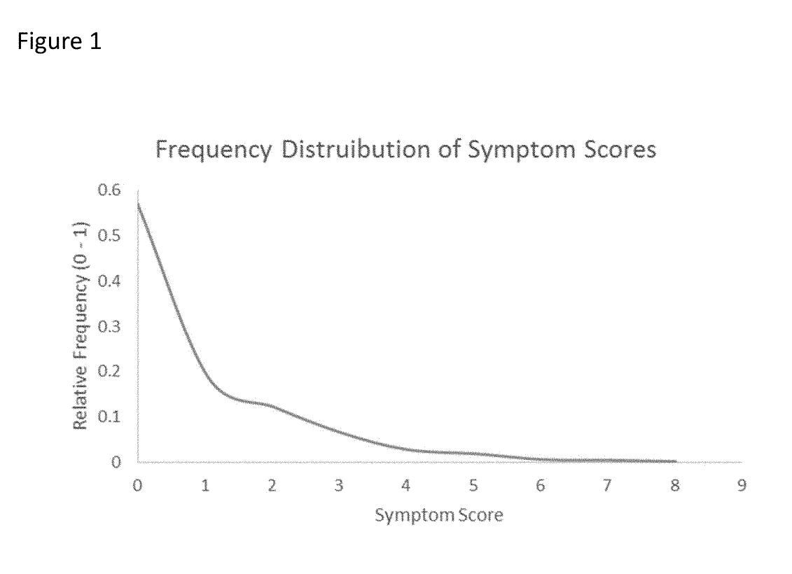

The primary parameter used was Total Symptom Sum, or TSS (sum of all time points), in active eye vs placebo eye. The secondary parameter used was “Upon instillation” Cooling score. FIG. 1 illustrates a frequency distribution of all symptom scores, based on a histogram of the data. The curve shown in the graph in FIG. 1 is skewed left, and does not approximate a normal distribution (bell curve). As such, nonparametric statistical analysis was used to determine if differences exist between treatments (menthol vs placebo). In the following results, a Wilcox Signed Rank test was employed to test the null hypothesis that Menthol and Placebo elicit statistically similar symptom scores on the Cooling Scale. If T_{Test Stat}<T_{Critical Value}. If so, then the null hypothesis can be rejected with 95% confidence.

### Results

Initial results from Day 1 of dosing indicated the Menthol-induced symptom response was statistically similar to Placebo in both Dry Eye and Healthy groups at the following concentrations:

0.000001%

0.00001%

However Menthol-induced symptom response was significantly higher than Placebo in both Dry Eye and Healthy groups, at the following concentration (Note that, although both groups could distinguish menthol from placebo at this concentration, Dry Eye TSS was significantly higher than Normal):

0.001%

Interestingly, at 0.0001% Dry Eye had significantly higher Menthol-induced symptom response than placebo, whereas Normals did not. From this data, it is evident that the concentration jump from 0.00001% to 0.0001% needed further investigation, to determine a more precise concentration at which dry eye could differentiate menthol from placebo but normals could not.

A range of 3 dilutions spanning this ten-fold concentration increase were created (0.000025%, 0.00005%, 0.000075%), and tested again in the same population, using the same dosing design (randomized eye, contralateral with placebo, 15 μl pipette instillation, 45 minute washout intervals). All concentrations tested are shown in Table 2, below, along with statistical significance indicated for either group according to a Wilcox Signed Rank Test (Total Symptom Sum, Menthol vs Placebo):

TABLE 2
Listing of concentrations and populations for which menthol elicited
a significantly higher symptom response than placebo (TSS), according
to a Wilcox signed rank test with 95% confidence.

| | Dry Eye Population | Healthy Population |
|---|---|---|
| | Is Menthol symptom | Is Menthol symptom |
| | response significantly higher | response significantly higher |
| Menthol | than placebo? | than placebo? |
| Concen- | ( ) = % of population that had | ( ) = % of population that had |
| tration | significantly higher menthol | significantly higher menthol |
| (w/v) | TSS than placebo TSS | TSS than placebo TSS |
| 0.000001 | NO (0%) | NO (0%) |
| 0.00001 | NO (0%) | NO (0%) |
| 0.000025 | NO (0%) | NO (0%) |
| 0.000050 | YES (75%) | NO (16%) |
| 0.000075 | YES (25%) | NO (16%) |
| 0.0001 | YES (25%) | NO (0%) |
| 0.001 | YES (75%) | YES (50%) |

From this table it is evident that the Dry Eye population can differentiate Menthol from Placebo at concentrations lower than Normal (a 50-fold lower concentration). In a previous study, it was demonstrated that at 0.01% concentration, Dry Eye has significantly higher symptom response than Normal patients. The findings shown above suggest that this concentration could be lowered drastically for a more precise diagnostic tool.

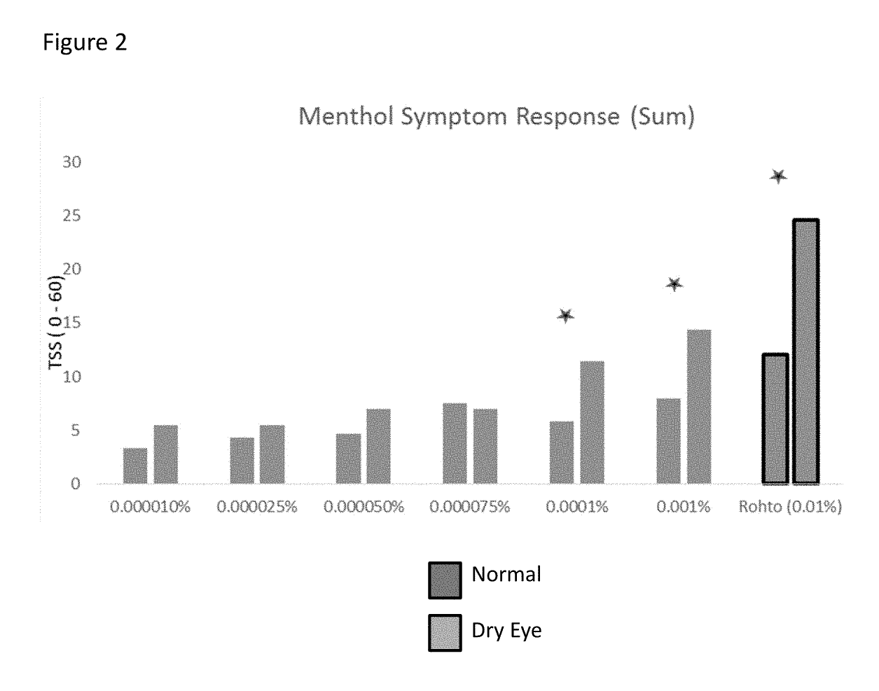

In addition to the Dry Eye population being able to differentiate menthol from placebo at a lower concentration than their normal counterparts, symptom response to menthol (TSS) was significantly higher in dry eye subjects at concentrations of 0.0001% and up, as shown in FIG. 2.

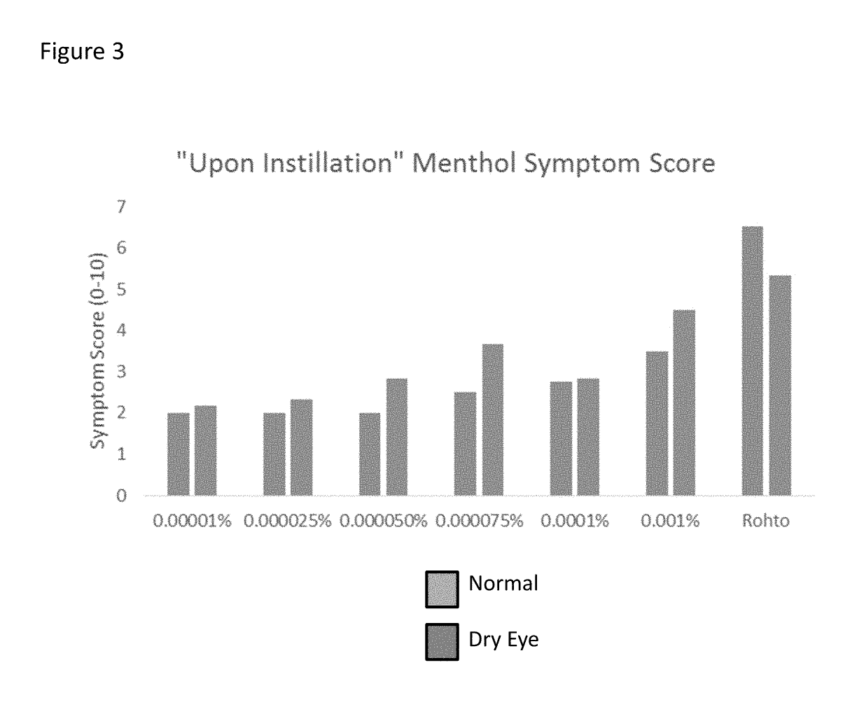

These results show that TSS provided a much better separating metric between dry eye and normal groups than did “Upon Instillation” symptom scores (FIG. 3). FIG. 3 shows the “Upon Instillation” menthol symptom response in Normal and Dry Eye populations. No significant differences were found at any concentrations tested.

At a concentration of 0.001%, menthol-induced TSS displayed strong correlations with Ora Calibra 4-symptom questionnaire, OSDI, and corneal staining (fluorescein).

TABLE 3
Correlation scores between Menthol TSS
(0.001%) and other study parameters

| | 4-Symptom | | Fluorescein Staining |
|---|---|---|---|
| | Questionnaire | OSDI | (Corneal) |
| Correlation | 0.64 | 0.79 | 0.50 |

The results provided above demonstrate the value of the TRPM8 agonist Menthol as a screening tool for Dry Eye studies. At the tested concentration of 0.0001% (1/100^{th} concentration of ROHTO Hydra) and higher, the dry eye group displayed significantly higher TSS than normals. In a more robust population of 48 subjects, ROHTO Hydra (0.01% Menthol), the dry eye group displayed significantly higher TSS (two-fold higher) than normals (n=33 dry eye, 15 normals). This data shows strong potential as an effective, yet inexpensive and easily conducted, screening tool for dry eye studies. This methodology may provide a less expensive alternative to the use of “run in” periods to exclude patients which are symptomatic responders to placebo or subjects that fail to demonstrate sufficient environmental symptoms. Additionally, this screening tool is valuable value for trials which seek to utilize therapies that are thought to act on this pathway.

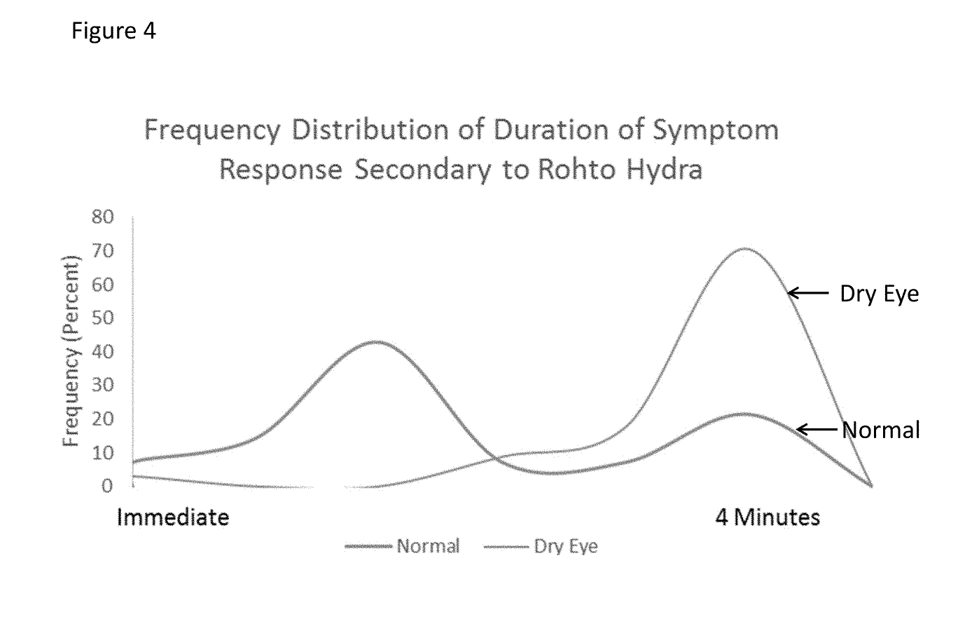

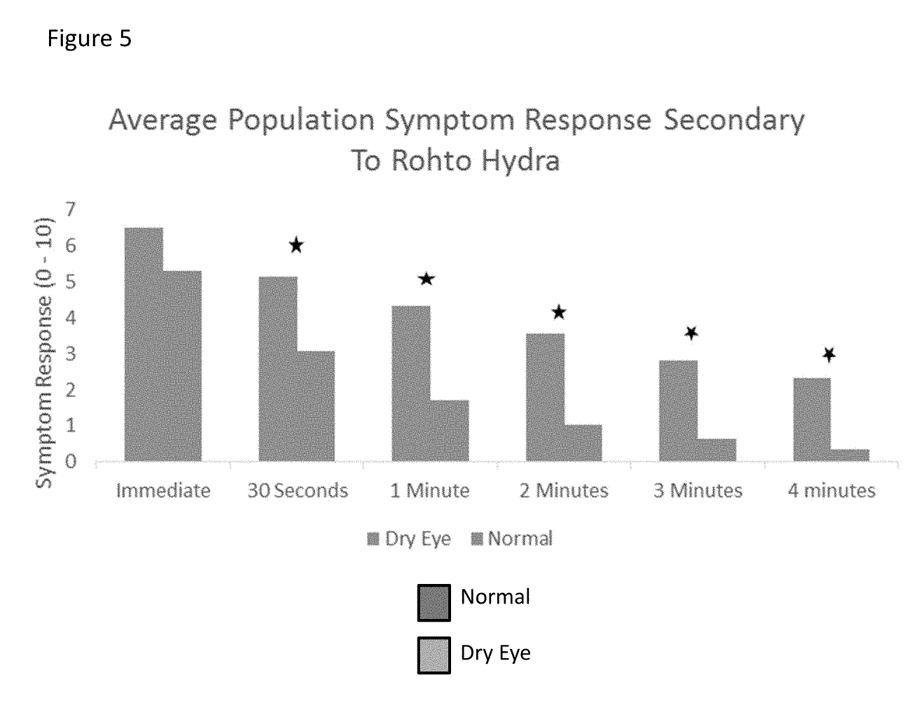

As shown in the data presented herein, duration of menthol-induced symptoms appears to be a good separator of normal and dry eye groups. In this pilot, the dry eye group showed a significantly longer duration of symptoms than the normal group secondary to Menthol instillation (p=0.004), with duration defined as the furthest time point post-dose which elicited a symptom score higher than 0 (Dry Eye mean=2.58 minutes, Normal mean=1.57 minutes, average of all concentrations). This same concept was used in a further study (see Example 3), and similar results were found with ROHTO Hydra (Dry Eye mean=3.6 Minutes, Normal mean=2.38 minutes, p<0.001). Frequency distribution of furthest time point which elicited a symptom response higher than 0 is plotted in FIG. 4. FIG. 5 shows that the separation in symptom scores becomes more clear with time, in dry eye versus normal populations. Each difference, with the exception of “Immediate”, is significantly higher in the Dry Eye group relative to the Normal group.

### Example 2

### Symptom Response and Modifiability of the Tear Film to Menthol

The goal of this study was to investigate subjective symptom response to a known concentration of Menthol, and also to examine thermal properties of the tear film, and its modifiability as a function of menthol, artificial tear, and environment.

Menthol, acting as a potent agonist of TRPM8, can be used to test this theory. We expect dry eye patients to display a more severe symptom reaction to a TRPM8 agonist, due to lower firing threshold. To further explore this idea, two populations of dry eye patients are included, defined by their symptoms response, or non-response, to a mucin-secreting agent in the controlled adverse environment (CAE). It is hypothesized that these two groups may represent two distinct subtypes of dry eye disease and given their distinct differences in symptoms response patterns may be phenotypically different in corneal nociceptor sensitivity.

The following experiments were performed using, but not limited to, the methods descried below.

### Methods

48 patients completed this IRB-approved study, spanning 3 populations that include Dry Eye Responders (to the Mimetogen drug), Dry Eye Non-Responders, and Normals. Study design surrounding Menthol symptom response utilized a masked, bilateral, crossover, randomized order design, with SYSTANE Ultra acting as placebo due to similarities in ingredients and viscosity to 0.01% Menthol w/v. A one hour wait interval between doses was implemented to allow full washout of drop and full recovery of TRPM8. 15 μl OU pipette dosing was employed for consistent dose volume, and subjects were asked to grade the “cooling” sensation of each eye separately on a scale of 0-10, where 0 is “no cooling” and 10 is “maximum cooling”, at the following time points: Upon instillation, 30 seconds, 1 minute, 2 minute, 3 minutes, and 4 minutes. Ora CALIBRA Ocular Discomfort and 4-symptom questionnaire was asked at the 5-minute post-dose time point. Corneal sensitivity was measured at approximately 10 minutes post-drop, and at baseline.

### Results

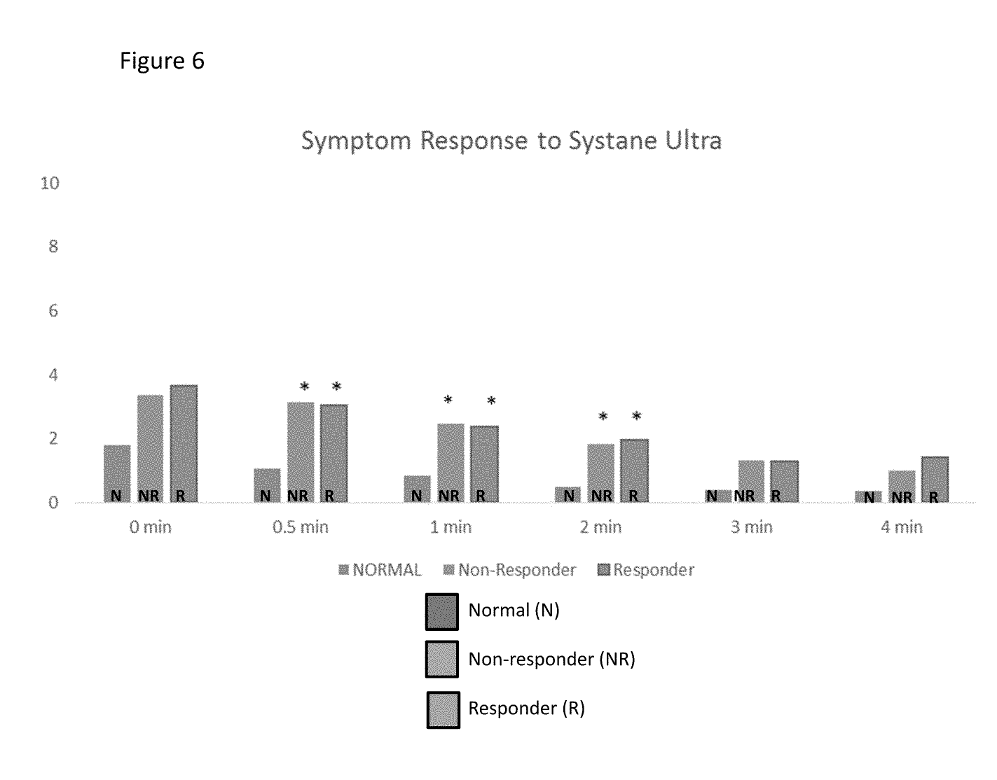

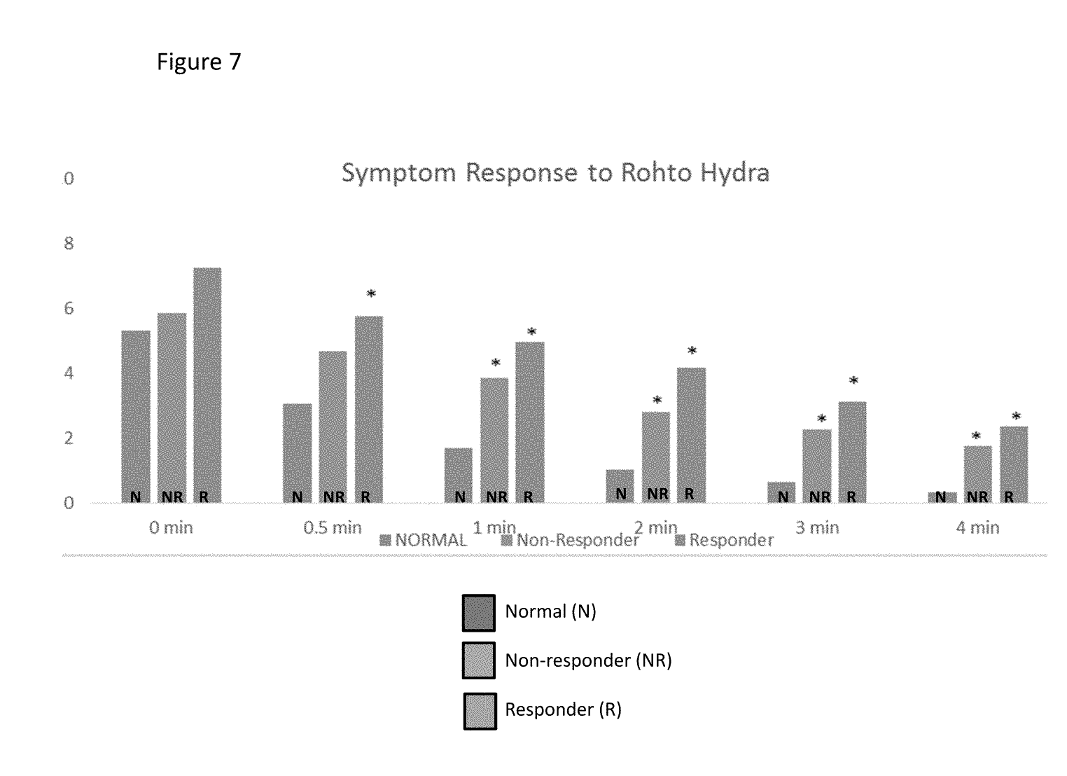

Symptom response to SYSTANE Ultra is shown in FIG. 6. T-tests were run to compare differences between populations, significant differences (p<0.05) are marked with an asterisk. No significant differences were found between the two dry eye populations, all asterisks denote a significant finding relative to the normal population. Symptom response to ROHTO Hydra is shown in FIG. 7. No significant differences were found between the two dry eye populations, all asterisks denote a significant finding (p<0.05) relative to the normal population.

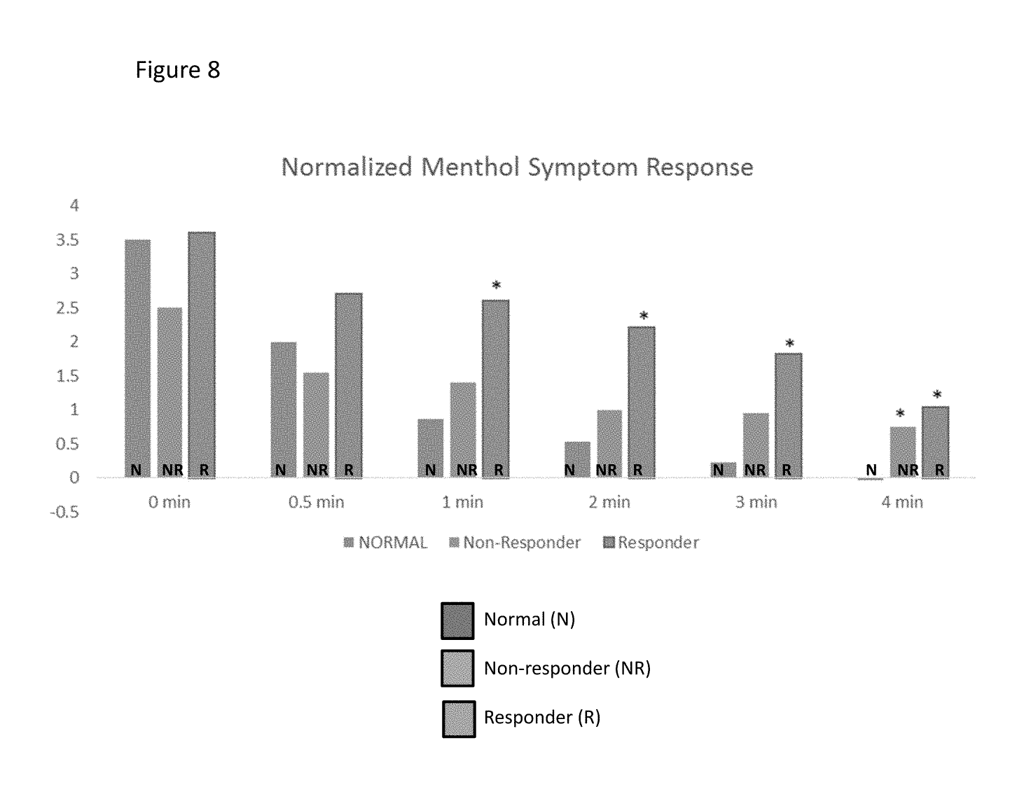

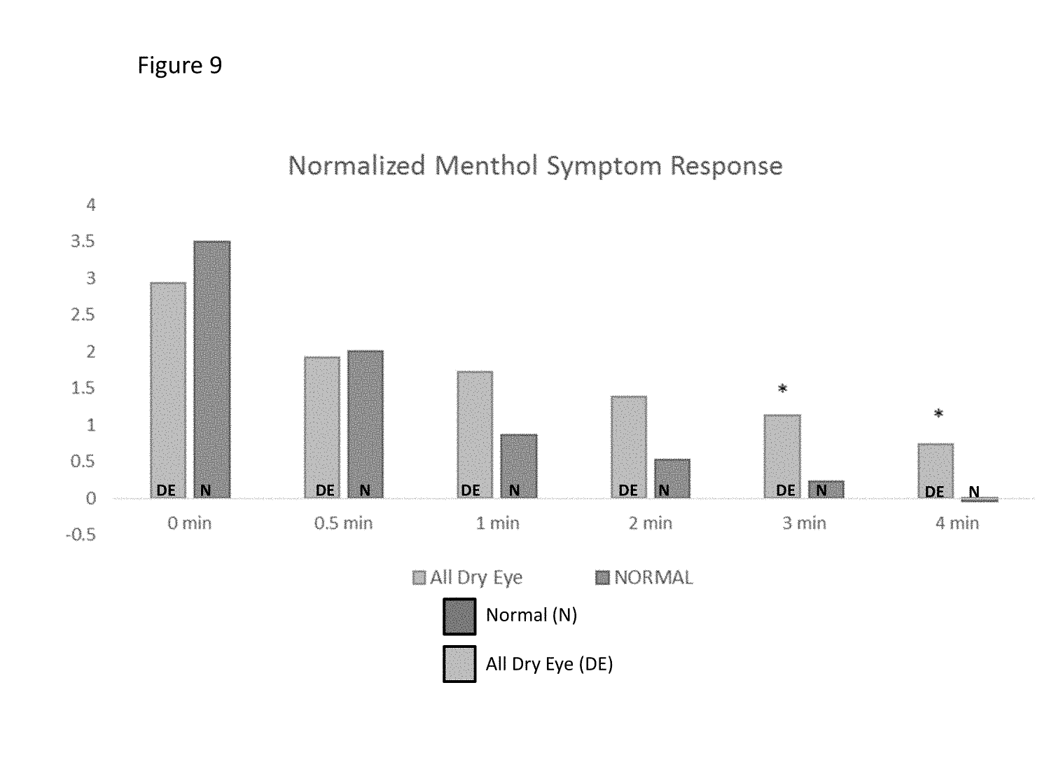

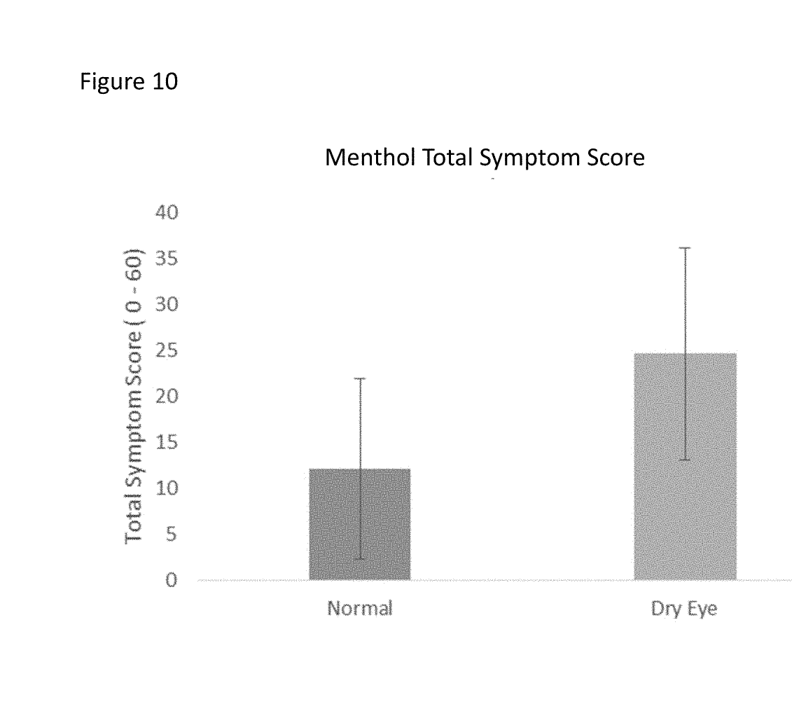

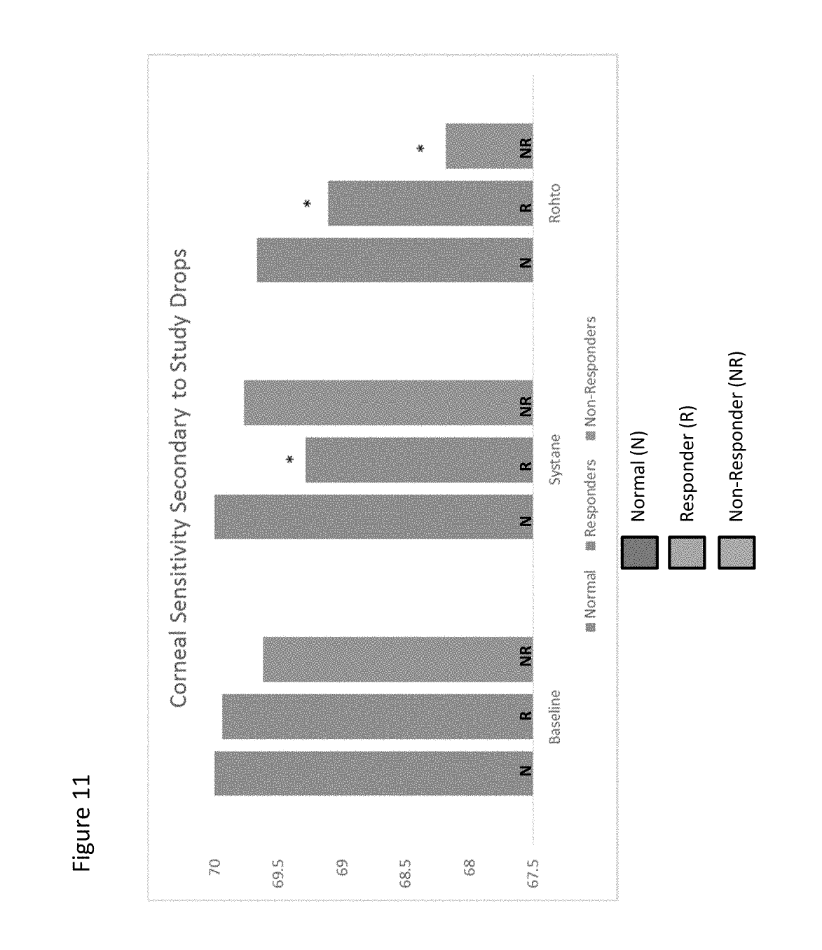

Normalized symptom response (ROHTO Hydra symptom response minus SYSTANE Ultra symptom response) is shown in FIG. 8. No significant differences were found between the two dry eye populations, all asterisks denote a significant finding (p<0.05) compared to the normal population. Normalized Menthol symptom response is shown in FIG. 9, as ALL dry eye patients vs normal (same y-axis scale as FIG. 8). Menthol TSS (Total Symptom Score) is the sum of all cooling time points (immediate, 30 s, 1 min, 2 mins, 3 mins, and 4 mins post-dose), averaged over OD and OS. FIG. 10 shows TSS for Normal and Dry Eye populations. Corneal sensitivity was measured as baseline, and approximately 10 minutes after each dose. Results are shown in FIG. 11.

The results show that significant differences were found between dry eye populations and age-matched normals The clear pattern of symptom progression over the 4-minute symptom query interval is distinct between dry eye and normal populations. While the initial and 30 second symptom responses displayed no significant differences between dry eye and normal populations, clear separation was found at 1, 2, 3 and 4 minutes post ROHTO Hydra. The use of menthol as a screening agent may provide a cost-efficient and accurate way to differentiate dry eye from normal patients.

It is important to note that the placebo used, SYSTANE Ultra, is not a true control in the sense that it too will act as a mild TRPM8 agonist, due to TRPM8 also being activated by liquid at room temperature. In fact, we do see a separation between dry eye groups and normal population secondary to SYSTANE Ultra (see FIG. 6), which is likely a result of this phenomena. Although both SYSTANE Ultra and ROHTO Hydra were stored at the same temperature, it is important to keep this property in mind. A possible way to circumvent this issue would be to replace ROHTO Hydra in this study design with an array of decreasing temperature SYSTANE Ultra, thus activating TRPM8 via cold activity and not via an agonist. However, the difficulty in controlling for exact temperature across all patients and time points poses a challenging clinical design, and utilizing the current design achieves superior control.

The extended duration of menthol symptom response found in the dry eye populations, compared to age-matched normals, may represent a clinically significant finding.

If an in-tact and functional neural feedback loop (normal patients) is assumed, it is then expected that TRPM8 activation will lead to an increase in basal tear secretion, as has been supported in mice models (Nociceptors (2011); Meng (2010); Meng et al. (2012)). Alternatively, with a compromised neural feedback loop (dry eye patients), TRPM8 activation would have a relatively minimal effect on lacrimation. As such, with equal concentration of menthol dosed across population, one can expect a lacrimation-inducing effect in normal patients, and not in dry eye patients. Several mice models have demonstrated that TRPM8 activation via menthol causes acute (up to 10 minutes) TRPM8 activation shift to warmer thresholds (Kurose, M., & Meng, I. D. (2013); Julius et al. (2002)). This effectively will cause post-dose TRPM8 activation to occur earlier in the tear cooling/evaporation cycle. If normal patients experience a lacrimation response to menthol, causing an increase in tear volume, activation of TRPM8 via tear film cooling/evaporation will occur later in the tear film cycle relative to dry eye patients. This could have the effect of TRPM8-based symptom trigger not being activated within an inter blink interval (IBI), as the increased tear volume acts as an enhanced temperature buffer to external air. If dry eye patients indeed do not experience the lacrimation-inducing effects of menthol due to compromised neuronal loop, the threshold TRPM8 activating temperature will occur more rapidly post-blink, and therefore elicit a more severe symptom response in duration.

The theory postulated above should be demonstrable via ocular thermodynamics secondary to TRPM8 activation. Due to activated TRPM8 receptors via Menthol, all patients should have symptomology earlier post-dose relative to baseline. In normal patients however, the increase in basal tear secretion may allow a longer time period before TRPM8 is activated again by cold temperature from ambient air (enhanced tear film volume equates to longer time to thermally equilibrate to cool ambient air). These conflicting forces may have an overall trend to keep percent temperature drop of the tear film within each IBI relatively similar to baseline. However, in dry eye patients, the lack of basal tear response to menthol will leave the tear film relatively unchanged. The tear film will reach a critical “TRPM8-activating” threshold earlier relative to baseline (due to Menthol causing an activation shift to warmer temperatures). This should have a net effect of decreasing the percent drop in tear film temperature within an IBI. If indeed this is the case, drug development surrounding the use of a TRPM8 antagonist to treat dry eye symptoms is certainly warranted. If sensation-induced activation of TRPM8 due to low tear volume is unable to induce lacrimation in dry eye patients, blocking this channel may provide a means of ceasing symptoms, while likely not impacting tear secretion due to an already compromised neuronal loop.

ROHTO Hydra appears to lower corneal sensitivity in both dry eye populations, but not in the normal population. This may be the result of menthol's analgesic properties via k-opioid receptor binding (Ghelardini, C. et al. (2002)). The analgesic effect may be too minimal to have an effect on normal, healthy eyes, with greater sensitivity than dry eye patients. Dry eye patients, with reduced corneal sensitivity to begin with (Belmonte, C. et al. (2005)), could experience a more substantial analgesic effect secondary to menthol, relative to baseline. Analgesic effects in normals may be difficult to capture via 70 mm cochet bonnet, but may need a more sensitive method to detect (baseline, post ROHTO Hydra sensitivity may drop, yet still be above 70 mm detectable).

Taken together, menthol appears to show value as a screening tool and a diagnostic for dry eye disease. The ease of administration and low-cost further warrant its use as such. Further exploration of tear film thermodynamics secondary to menthol was accomplished, and the results will provide further insight into the role of TRPM8 in dry eye, and the role of TRPM8-acting agents in dry eye therapy.

### Example 3

### Dynamic Sensitivity of Afferent Corneal Cool-Sensitive Thermoreceptors to Menthol in Dry Eye Patients

Given that Dry Eye Disease (DED) can result in enhanced corneal sensitization, and that menthol elicits a neuronal response similar to mild cooling temperatures, it was hypothesized that DED patients would display an enhanced corneal cooling response compared to healthy controls. Therefore, the objectives of this study were to evaluate the degree to which the cooling sensation of menthol-containing eye drops (ROHTO Hydra, 0.01% Menthol w/v (The Mentholatum Company)) varies between DED and Normal patients compared to active placebo (SYSTANE Ultra (Alcon Laboratories, Inc.)) and to evaluate the time course of cooling sensitivity to both post-drop instillation and as a function of duration of disease. This is the first study in humans to investigate these outcomes in DED patients.

The following experiments were performed using, but not limited to, the methods descried below.

### Methods

The informed consent and study protocol were approved by a properly constituted Institutional Review Board (Alpha IRB, San Clemente Calif.) regulations and the study was conducted in accordance with the Tenets of the Declaration of Helsinki. This was a single-center, open-label, single blind, bilateral design where subjects were randomly assigned to initially receive either 15 μl drops of either ROHTO Hydra containing menthol followed by SYSTANE Ultra artificial tear by pipette and a sterile tip (OU). Following a washout period of one hour, the drops were instilled again in reverse order.

All subjects provided written informed consent and were at least 18-years of age of any gender or race. Subjects underwent baseline slit-lamp examination and Early Treatment Diabetic Retinopathy Study (ETDRS) visual acuity and provided ocular medical/surgical history. DED subjects had a documented history of disease and Normal subjects had no prior diagnosis of DED, no history of artificial tears for the previous month for ocular dryness, and had scores <2 (on a 0-5 scale).(Meerovitch, Invest. Ophthalmol. Vis. Sci. 2013; 54(15):43432013).

Subjects were excluded from the study if they had contraindications, allergies or sensitivities to products used (ROHTO Hydra or SYSTANE Ultra), had any active ocular infectious or inflammatory diseases that required therapeutic treatment, had uncontrolled systemic disease, had a history of LASIK surgery in either eye or had undergone any ocular surgery within the previous 12-months or currently had punctal plugs in either eye. Contact lens wear was prohibited for 30-days prior to study. Subjects were required to avoid prescription ophthalmic medications, over-the-counter solutions including artificial tears for 24-hours prior to the study visit. Subjects were not permitted to have been enrolled in another investigational drug or device trial within 30-days of the study and were not pregnant, planning to become pregnant or nursing an infant during the study.

Outcomes

The severity of ocular coolness sensation of each eye was evaluated after the instillation of study medication containing menthol or control (either ROHTO Hydra or SYSTAIN Ultra) at 0, 0.5, 1, 2, 3, and 4 minutes using an 11-point (0 to 10) scale, where 0=not cool and 10=very cool (this is referred to as the “0-10 Cooling Scale” herein). The sum of the six time points generated a cooling sum score (0 to 60) and was averaged across subjects to generate a mean sum cooling score. Cooling sensation was evaluated as a function of age and the duration of DED (patients having DED greater than or less than 10-years).

Dry Eye Assessments

Following instillation of study drug and after administration of the cooling scale, objective signs and subjective symptoms of DED were collected. Objective measures included: 1) The anesthetized Shirmer's test was administered to evaluate tear volume. (Schirmer, Graefes Arch Clin Exp Ophthalmol. 1903; 56:197-291).

2) Tear film break-up time (TFBUT) was assessed by instilling 5 μL of fluorescein dye into the tear film and measuring the time between the last complete blink to the first appearance of tear film break-up.

3) Corneal fluorescein (inferior, superior, central regions) and conjunctival lissamine staining (nasal and temporal regions) were utilized to visualize the effects of desiccation on the ocular surface and were scored on a standardized 0-4 scale (0=none to 4=confluent staining, half-point units allowed). (Meerovitch, 2013). Each region was graded separately; the sum of the three corneal regions generated a corneal sum score (0-12), and the sum of the two conjunctival regions generated a lissamine sum score (0-8). Assessments were made 3-5 minutes after drop instillation.

Subjective symptoms of ocular discomfort, burning, dryness, grittiness, and stinging were scored on a standardized 0 to 5 point severity scales (no half-point grades). Each symptom was graded separately; the sum of the 5 symptoms generated a symptom sum score (0-25) and was averaged across subjects to generate a mean sum symptom score.(Meerovitch, 2013).

Safety analyses included slit lamp biomicroscopy, BCVA, and adverse event query.

Statistical Analyses

Descriptive statistics were calculated for all variables. For population comparisons an unpaired t-test was used to compare mean differences in ocular cooling sensation between groups at each timepoint. Eyes within a subject were averaged to obtain one measure per subject at each time point. Post hoc analysis utilized a paired t-test to compare mean differences in cooling scores between groups of DED patients (less than 10-years vs. greater than 10-years). For all statistical comparisons, a p-value less than 0.05 (2-sided) was considered statistically significant. All calculations were performed using Microsoft Office Professional Plus Excel (10).

### Results

Demographics

The mean age of the DED group (N=33) 62.2±8.6 years (range 33-76 years), was significantly older than the normal group (N=15) was 53.5±7.6 years (range 36-63 years). There were 25 females and 8 males in the DED group and 8 females and 7 males in the normal group.

Corneal Cooling Severity

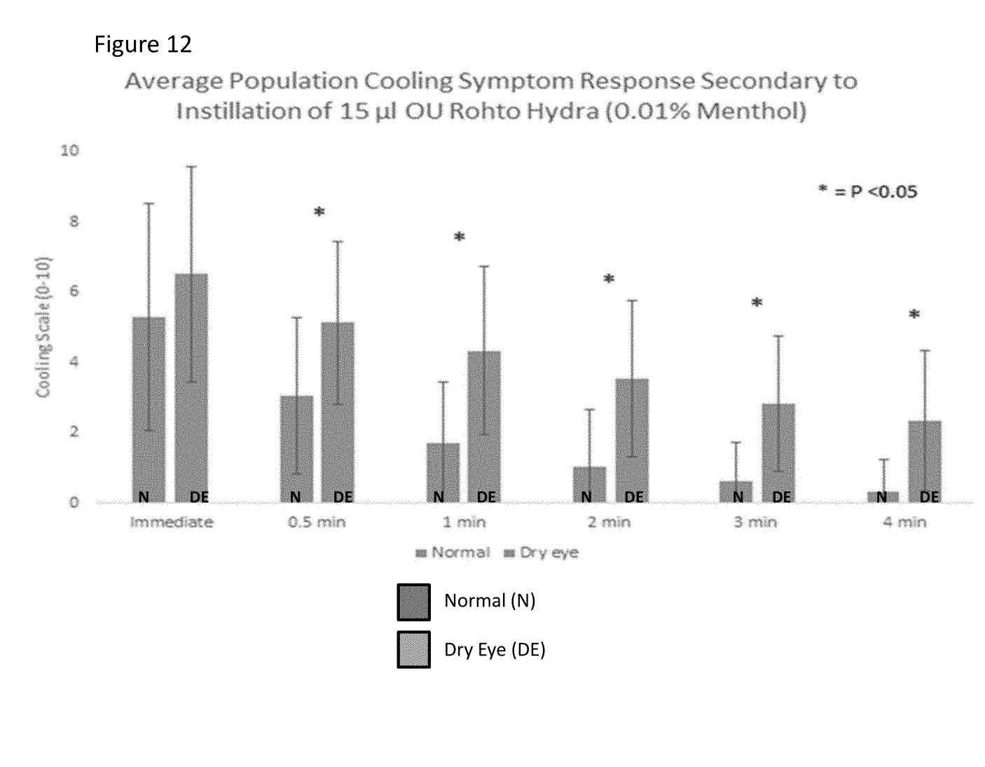

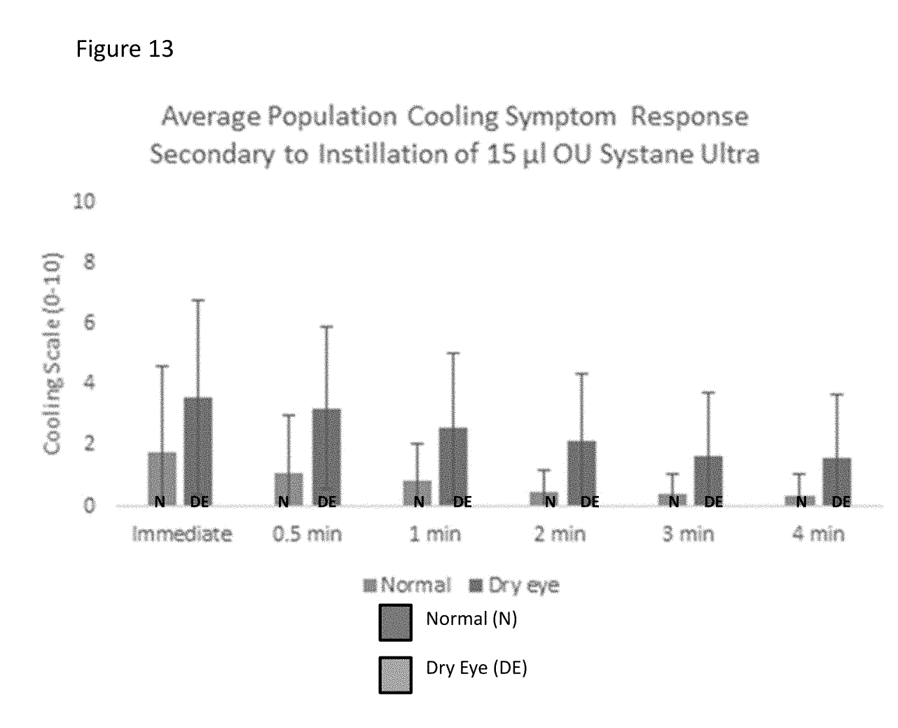

The mean (both eyes) cooling scores (FIGS. 12 and 13) were significantly greater in the DED group than in the normal group at 0.5, 1, 2, 3, and 4-minutes post-dose to both ROHTO Hydra (p=0.006, p=0.0004, p=0.0003, p=0.0002 and p=0.0007, respectively) and SYSTANE Ultra artificial tear (p=0.007, p=0.01, p=0.006, p=0.02, and p=0.03, respectively).

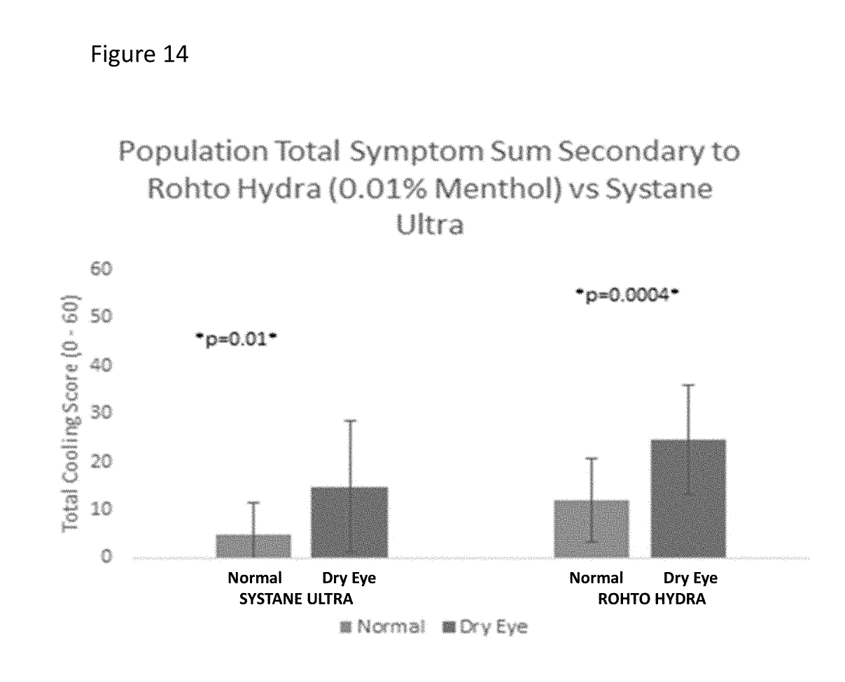

In FIG. 14, the mean of the sum cooling scores, (0-10 scale, 6 time points, range 0-60) was significantly greater in the DED group compare to the normal group after ROHTO Hydra installation (p=0.0004) vs. SYSTANE Ultra (p=0.01).

A post hoc subgroup analysis revealed that the mean total cooling score post-ROHTO Hydra application was significantly greater in patients who reported having DED for less than 10-years (N=17) compared to those who reported having DED greater than 10-years (N=16) (Table 1, p=0.04). No significant differences in the objective and subjective assessments of DED were observed between these two groups, as shown in Table 4, below. There were no carryover effects detected in these variables as a result of the crossover study design (order of drug administration).

TABLE 4
Mean (±SD) signs and symptoms scores in patient-reported DED fewer than 10-
years (N = 18) vs. those patient-reported DED greater than 10-years (N =
15). Comparisons between groups were calculated using the Student t-test. No
significant differences were found for any of the variables. Groups are age-matched.

| | | Fluorescein | Lissamine | | Keyword |
|---|---|---|---|---|---|
| Duration | Anesthetized | Staining | Staining Sum | | Symptom |
| of DED | Schirmer's | Sum Score | Score | TFBUT | Sum Score |
| DE Patients >10 | 6.233 | 1.523 | 1.35 ± 0.93 | 2.838 | 8.412 |
| years (n = 15) | | | | | |
| DE Patients <10 | 4.861 | 2.292 | 0.93 ± 1.33 | 3.06 | 10.556 |
| years (n = 18) | | | | | |
| p-value | 0.50 | 0.14 | 0.38 | 0.39 | 0.17 |

Maintaining the integrity of the tear film is a constant challenge to those suffering from DED due to evaporation. However, considerable difficulties exist in assessing causality to the development of DED due to the variability of signs and symptoms resulting from the confounding variables of lifestyle, environment, and a multitude of compensatory mechanisms that may mask the underlying disease.(Foulks, Ocul Surf. 2003 July; 1(3):107-26). In this study, the sensitivity of the cooling sensation response of the afferent limb of the lacrimal functional unit in DED patients in relation to standard assessments of DED was examined.

The present study demonstrated a clear progression of the subjective sensation of cooling over 4-minutes post-installation to menthol as well as to artificial tears (active control) in DED patients compared to normal subjects (FIGS. 12 and 13). It is not surprising that the placebo groups displayed a cooling response as the SYSTANE Ultra (and ROHTO Hydra) solutions were stored at a controlled room temperature (˜20 ° C.) which was cooler than the average corneal temperature in man (˜34.3° C.±0.7).(Efron, Curr Eye Res. 1989 September; 8(9):901-61989). When the population scores were summed, the magnitude of the cooling response to ROHTO Hydra and SYSTANE Ultra were significantly greater in the DED group than in the control group (FIG. 14).

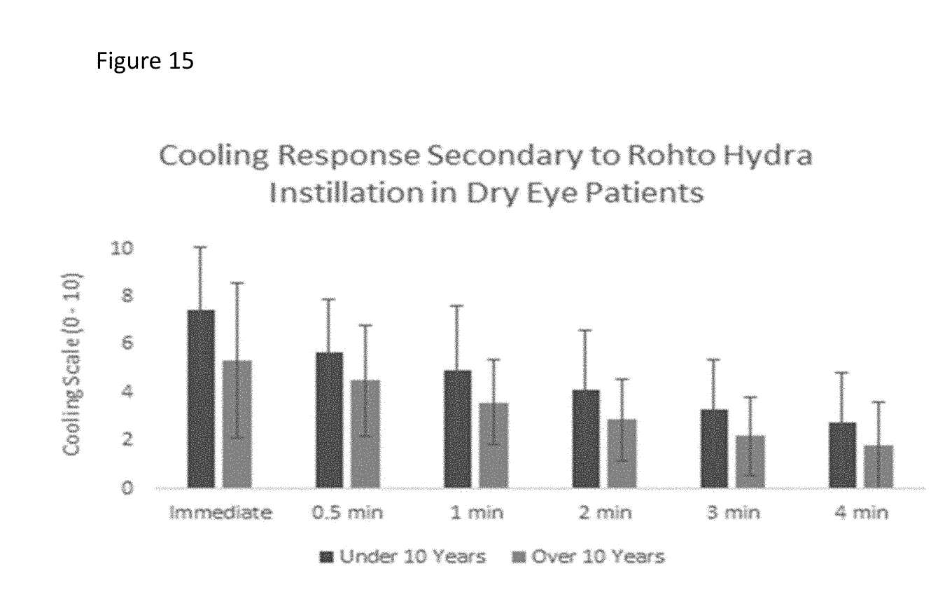

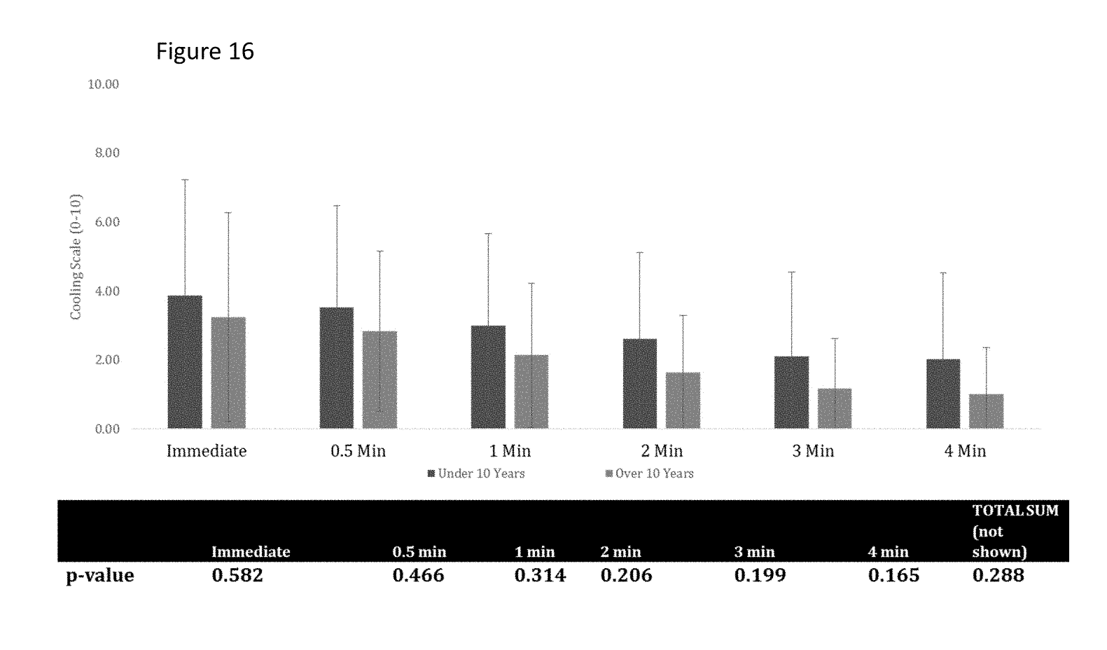

An important finding of this study was revealed from subgroup analysis that demonstrated that the overall sensation of cooling was greater in subjects who self-reported having DED for less than 10-years compared to those patients who self-reported having DED for greater than 10-years, as shown in Table 5 and Table 6, below, and in FIG. 15. Further, as shown in FIG. 16, when the SYSTANE cooling response was examined in under 10 years and over 10 years diagnosis DED groups no significant differences were found at any time point.

TABLE 5
Mean (±SD) of the sum cooling scores (0-10 scale, 6 time
points, range 0-60) post-ROHTO Hydra application in patient-
reported DED fewer than 10-years (N = 18) vs. those
patient-reported DED greater than 10-years (N = 15).
Comparisons between groups were calculated using the Student
t-test (p < 0.05). Groups are age-matched.

| | | | Mean Population |
|---|---|---|---|
| | | | Total Cooling |
| Group | N | Age | Score (±SD) |
| DED Over 10 Years | 15 | 61.73 | 20.2 ± 10.69 |
| Duration | | | |
| DED Under 10 years | 18 | 62.88 | 28.28 ± 10.93 |
| Duration | | | |
| p-value | — | =0.72 | =0.04 |

TABLE 6
Mean cooling scores post-ROHTO Hydra application as a
function of time in patients who reported having DED
for less than 10-years (N = 18) vs. those having
DED greater than 10-years (N = 15). Groups are age-matched.

| | | | | 0.5 | 1 | 2 | 3 | 4 |
|---|---|---|---|---|---|---|---|---|
| Group | N | Age | Immediate | min | min | min | min | min |
| DE Over | 15 | 61.73 | 5.36 | 4.5 | 3.6 | 2.87 | 2.2 | 1.79 |
| 10 Years | | | | | | | | |
| Duration | | | | | | | | |
| DE Under | 18 | 62.88 | 7.47 | 5.67 | 4.94 | 4.11 | 3.33 | 2.75 |
| 10 Years | | | | | | | | |
| Duration | | | | | | | | |
| p-values | — | =0.72 | =0.047 | =0.15 | =0.11 | =0.11 | =0.09 | =0.18 |

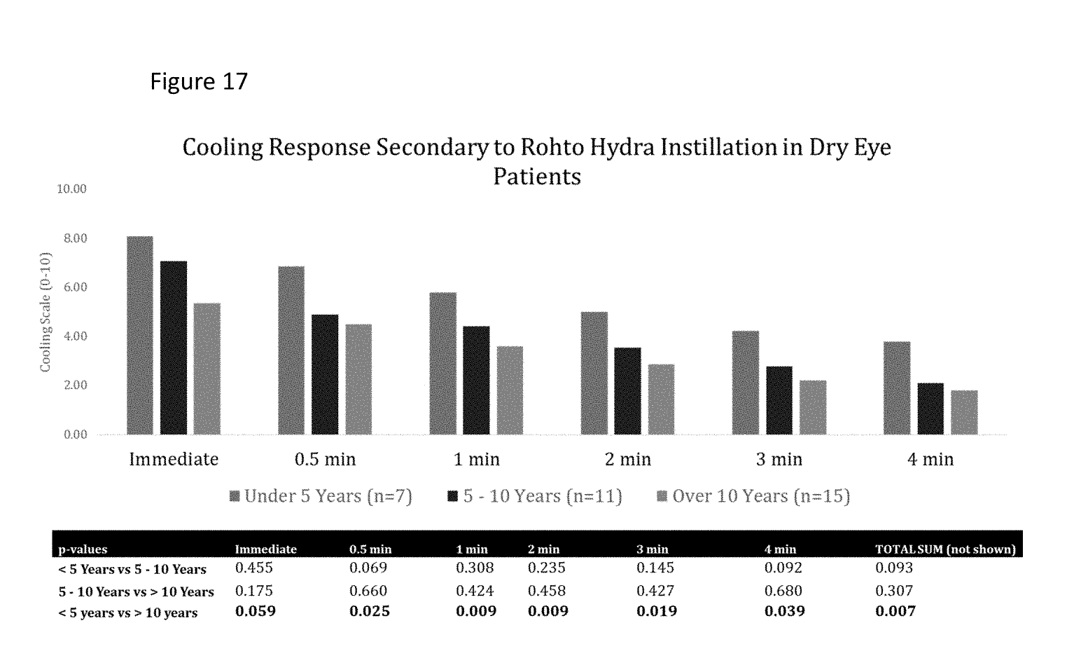

Next, cooling response secondary to ROHTO Hydra instillation in dry eye patients was examined in dry eye patient populations under 5 years from diagnosis, 5-10 years from diagnosis and over 10 years from diagnosis. The results are presented in FIG. 17, and show that there are no differences in the results between <5 and 5-10 year dry eye groups. However, differences at all time points between <5 and >10 years were found.

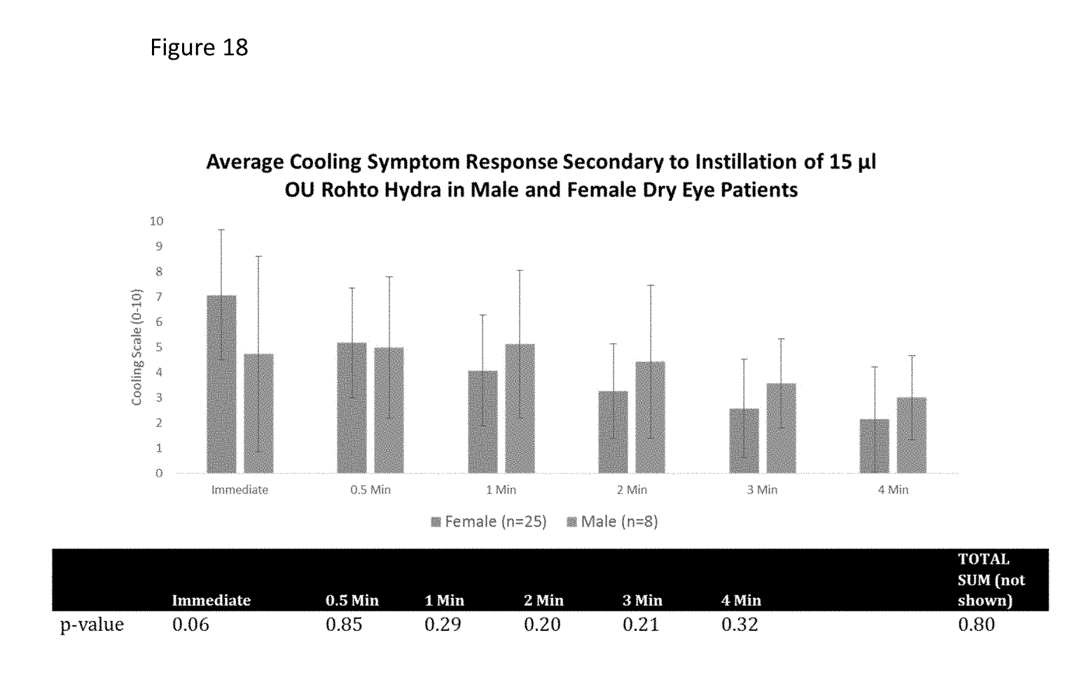

FIG. 18 shows the ROHTO cooling response in male vs female subjects with DED. Average cooling symptom response secondary to instillation of 15 μl OU ROHTO hydra in male and female dry eye patients was determined. As shown in FIG. 18, trending differences (not significant) were found at the immediate time point, and the results at all other time points for male and female patients are similar. Reverse trending was found at immediate, 0.5 min from other time points.

Table 7, shown below, summarizes Dry Eye Diagnosis duration vs other study signs. As shown in Table 7, no differences in study signs/symptoms were found in Dry Eye patients with >10 years of diagnosis history vs <10 years diagnosis history. Teat Film Break-up time (TFBUT) is the time after a blink that the tear film remains stable before it begins to break apart, measured in seconds. Symptoms Sum is achieved by combining all symptoms scores collected using the Ora Calibra symptoms scale, and is the sum of all 5 questions (possible range of scores is 0-25, each of the 5 questions is 0-5).

TABLE 7

| | | | | Corneal | Ora |
|---|---|---|---|---|---|
| | Anesthetized | Corneal | | Staining | Calibra |
| Duration | Schirmer's | Staining | | increase | Symptom |
| of DED | Score | Sum | TFBUT | in CAE | Sum |
| DE Patients >10 | 6.233 | 1.523 | 2.838 | 2.045 | 8.412 |
| years (n = 15) | | | | | |
| DE Patients <10 | 4.861 | 2.292 | 3.06 | 2.063 | 10.556 |
| years (n = 18) | | | | | |
| p-value | 0.499 | 0.135 | 0.388 | 0.971 | 0.167 |

Table 8, below, shows the ROHTO cooling response in Dry Eye subgroups vs other signs/symptoms. Dry Eye patients were divided based on Total Cooling Score (<25 or >25) and compared to other study metrics. As shown in Table 8, there were trending (not significant) differences in Schirmer's between dry eye patients with high (>25) vs low (<25) Total Cooling Score (aggregate) to ROHTO. Other signs/symptoms displayed no significance between groups.

TABLE 8
ROHTO Cooling Response in Dry Eye subgroups vs Other Signs/Symptom

| | | | | Corneal | |
|---|---|---|---|---|---|
| | | | | Fluorescein | Ora |
| | Anesthetized | Corneal | | Staining | Calibra |
| Total Cooling | Schirmer's | Staining | | increase | Symptom |
| Score Category | Score | Sum | TFBUT | in CAE | Sum |
| DE Patients <25 | 7.375 | 1.795 | 2.841 | 1.977 | 8.563 |
| Total Cooling Score | | | | | |
| (n = 16) | | | | | |
| DE Patients >25 | 3.706 | 1.955 | 3.054 | 2.136 | 10.588 |
| Total Cooling Score | | | | | |
| (n = 16) | | | | | |
| p-value | 0.063 | 0.769 | 0.430 | 0.748 | 0.207 |

Table 9, below, shows ROHTO Cooling Response in DE subgroups vs Other Signs/Symptoms. Individual ROHTO time points were compared to signs/symptoms in high vs low responders in the following slides (Immediate, 0.5 Min, 1 Min, 2 Min). No statistical differences at 3 or 4 minute time points in any category (data not shown). As shown in Table 9, Schirmer's Score was significantly different between Low (<5) and High (5-10) Cooling Score.

TABLE 9

| | | | | Corneal | Ora |
|---|---|---|---|---|---|
| | Anesthetized | Corneal | | Staining | Calibra |
| Immediate | Schirmer's | Staining | | increase | Symptom |
| Timepoint | Score | Sum | TFBUT | in CAE | Sum |
| DE Patients | 8.083 | 1.833 | 2.798 | 2.083 | 8.000 |
| Immediate | | | | | |
| Cooling Score <5 | | | | | |
| (n = 12) | | | | | |
| DE Patients | 4.000 | 1.904 | 3.051 | 2.083 | 10.524 |
| Immediate | | | | | |
| Cooling Score | | | | | |
| 5-10 (n = 21) | | | | | |
| p-value | 0.045 | 0.898 | 0.403 | 1.000 | 0.128 |

Table 10, below, shows ROHTO Cooling Response in DE subgroups vs. Other Signs/Symptoms (0.5-mins) As shown in Table 10, the Ora Symptom Sum was significantly different between Low (<5) and High (5-10) Cooling Score, Schirmer's.

TABLE 10

| | | | | Corneal | | Ora |
|---|---|---|---|---|---|---|
| | Anesthetized | Corneal | | Staining | | Calibra |
| | Schirmer's | Staining | | increase | | Symptom |
| 0.5 Minutes | Score | Sum | TFBUT | in CAE | ODSI | Sum |
| DE Patients | 8.000 | 1.464 | 2.886 | 1.857 | 15.083 | 7.500 |
| 0.5 Minute | | | | | | |
| Cooling Score <5 | | | | | | |
| (n = 12) | | | | | | |
| DE Patients | 4.048 | 2.067 | 2.976 | 2.150 | 15.714 | 10.810 |
| 0.5 minute | | | | | | |
| Cooling Score | | | | | | |
| 5-10 (n = 21) | | | | | | |
| p-value | 0.053 | 0.294 | 0.812 | 0.535 | 0.848 | 0.043 |

Table 11, below, shows ROHTO Cooling Response in DE subgroups vs. Other Signs/Symptoms (0.5-mins) As shown in Table 11, Ora Calibra Symptom Sum was significantly different between Low (<5) and High (5-10) Cooling Score.

TABLE 11

| | | | | Corneal | | Ora |
|---|---|---|---|---|---|---|
| | Anesthetized | Corneal | | Staining | | Calibra |
| | Schirmer's | Staining | | increase | | Symptom |
| 1 Minute | Score | Sum | TFBUT | in CAE | ODSI | Sum |
| DE Patients | 6.938 | 1.205 | 2.842 | 1.841 | 14.938 | 7.563 |
| 1 minute | | | | | | |
| Cooling Score <5 | | | | | | |
| (n = 15) | | | | | | |
| DE Patients | 4.118 | 2.545 | 3.053 | 2.273 | 16.000 | 11.529 |
| 1 minute | | | | | | |
| Cooing Score | | | | | | |
| 5-10 (n = 18) | | | | | | |
| p-value | 0.157 | 0.055 | 0.487 | 0.217 | 0.555 | 0.014 |

To explore potential effects of last study drop on Schirmer's Score, analysis was conducted to see if order or treatment (ROHTO vs SYSTANE) impacted score. At least 20 mins post final drop, Anesthetized Schirmer's test with tear meniscus removed via sterile weck-cel after anesthetic instillation. The results are summarized in Table 12, and show that no significant differences in Schirmer's Score as a function of treatment order was found.

TABLE 12

| Order of Last Study Drop | Schrimer's Score |
|---|---|
| Systane before Schirmer's | 6.12 |
| Rohto before Schirmer's | 4.81 |
| p-value | 0.52 |

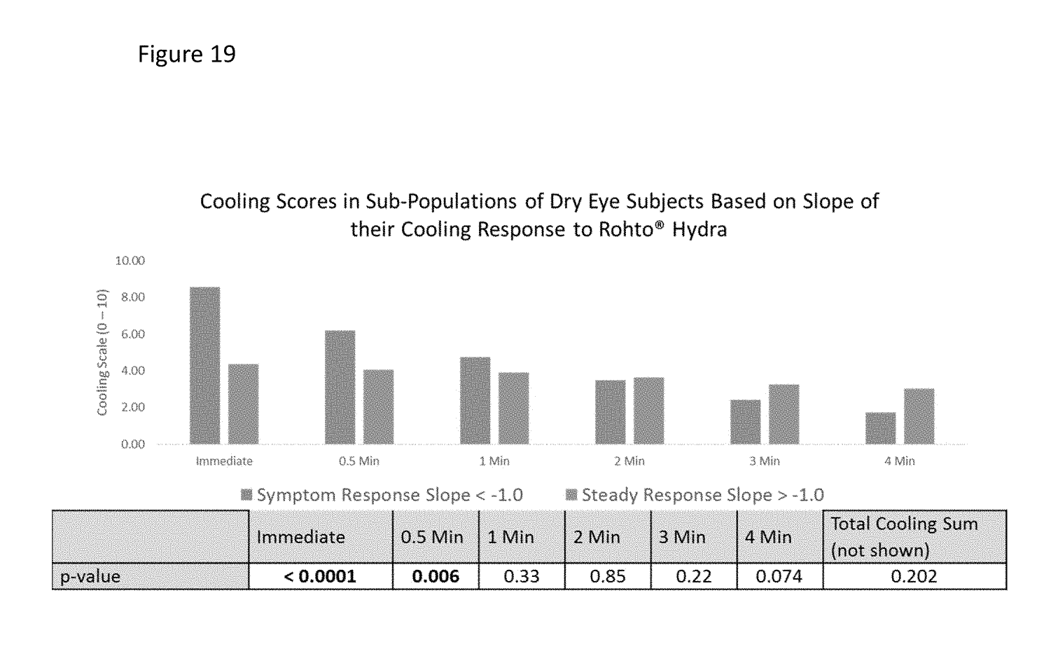

From these experiments, it appeared that two distinct “slopes” of ROHTO cooling response existed in Dry Eye patients, with one group having high initial cooling response and rapidly decaying and the other having relatively lower initial response and retaining the sensation over time. This is shown in FIG. 19. FIG. 19 shows the cooling scores in sub-populations of Dry Eye subjects based on slope of their cooling response to ROHTO Hydra.

Table 13 shows the slope of cooling response vs. other signs. High vs Low slope of cooling response was compared to other study metrics. As shown in Table 13, both symptom-based scores were significantly different between the two groups, yet all signs were statistically similar. Less than −1.0 slope represents a more negative slope (steeper downward slope, blue data on FIG. 19). Greater than −1.0 slope represents a less negative or a positive slope (flatter or increasing cooling score over time, orange data on FIG. 19).

TABLE 13

| | | Ora Calibra | | | |
|---|---|---|---|---|---|
| Category of | | Symptom | | | |
| Cooling Response | OSDI | Sum | TFBUT | Staining | schirmers |
| Symptom Response | 19.1 | 11.4 | 2.94 | 1.98 | 5.44 |
| Slope <−1.0 | | | | | |
| (n = 17) | | | | | |
| Steady Response | 11.7 | 7.8 | 2.96 | 1.75 | 5.53 |
| Slope >−1.0 | | | | | |
| (n = 16) | | | | | |
| p-value | 0.01 | 0.02 | 0.95 | 0.67 | 0.96 |

The data and results presented herein further suggests that the decreased cooling sensitivity in observed in the subgroup of patients with DED over 10-years is not based on differences in signs (corneal staining, TFBUT) and symptoms (symptom sum score) between groups but rather to duration of disease (Tables 4-7). Although the literature suggests that age, per se, is the primary cause of decreased corneal sensitivity in DED patients (De Paiva, Am J Ophthalmol. 2004 January; 137(1):109-154; Exp Eye Res. 2008 June; 86(6):879-85), this data shows for the first time in humans that the longer a patient lives with DED, the less sensitive the cornea becomes to cooling sensation. The reason for this is likely multifactorial. The specialized TRPM8 ion channels within first order cold thermoreceptors nerve endings that transduce thermal stimuli to the trigeminal brainstem (TBNC) play important role in monitoring ocular dryness and cooling (Exp Brain Res. 2009 June; 196(1):13-30). In humans, repeated application of menthol in non-ocular tissue significantly decreases the cold pain threshold and increases in pain sensitivity. (Eur J Pain. 2014 October; 18(9):1248-58. doi: 10.1002/j.1532-2149.2014.484.x. Epub 2014 Apr. 29). The results presented herein demonstrate that application of menthol to the cornea produces a heightened cooling response that is likely due to an enhanced heightened expression of TRPM8 receptors. Decreased cooling sensitivity in patients who have DED for extended periods of time may due to the prolonged effects of inflammation, neural injury, accessory gland dysfunction and other disease-specific factors, resulting in a less severe “cooling” sensation in compared to those who have had DED for shorter periods of time. The data support this postulation and provide a potential basis for the use of menthol as a simple yet powerful diagnostic tool and holds importance in the development of novel drug therapies for the management of DED.

### Example 4

### TRPM8 Mouse Studies

Experiments were performed to determine if different concentrations of TRPM8 can affect tear production in Balb/C mice. On day 0, 120 animals were split into 3 cohorts each containing 4 groups with n=10. For all 3 cohorts, data were analyzed as tear production, in millimeters, per 15 seconds. Two-way ANOVA was used to analyze the data where groups were compared to the group that received water. Another two-way ANOVA was used to analyze the data where the groups were being compared to their respective baselines. The results are as follows.

### Cohort 1

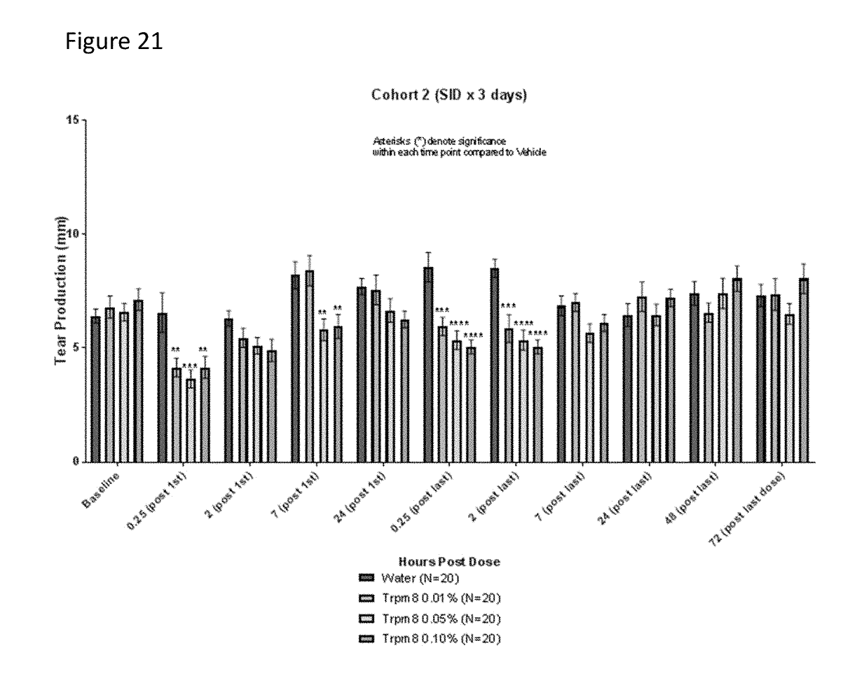

For Cohort 1, baselines were obtained on Days 10, 11 and 14. Tear production was measured for each mouse for 15 seconds per each eye. Mice received doses of either 0.1% TRPM8, 0.05% TRPM8, 0.01% TRPM8, or water once on Day 16. Mice had tear production measured at time points: 15 mins, 2 hours, 7 hours, 24 hours and 48 hours post dose. FIG. 21 shows the results for Cohort 1. Table 14, below, shows the results of two-way ANOVA compared to respective baseline for Cohort 1.

TABLE 14
Cohort 1 OU

| | |
|---|---|
| Trpm8 0.10% (N = 20) | Trpm8 0.01% (N = 20) |

| | | | |
|---|---|---|---|
| Baseline vs. 0.25 | ns | Baseline vs. 0.25 | *** |
| Baseline vs. 2 | ns | Baseline vs. 2 | ns |
| Baseline vs. 7 | ns | Baseline vs. 7 | ns |
| Baseline vs. 24 | * | Baseline vs. 24 | ns |
| Baseline vs. 48 | ns | Baseline vs. 48 | ns |

| | |
|---|---|
| Trpm8 0.05% (N = 20) | Water (N = 20) |

| | | | |
|---|---|---|---|
| Baseline vs. 0.25 | * | Baseline vs. 0.25 | ns |
| Baseline vs. 2 | ns | Baseline vs. 2 | ns |
| Baseline vs. 7 | ns | Baseline vs. 7 | ** |
| Baseline vs. 24 | ns | Baseline vs. 24 | ** |
| Baseline vs. 48 | * | Baseline vs. 48 | ns |

### Cohort 2

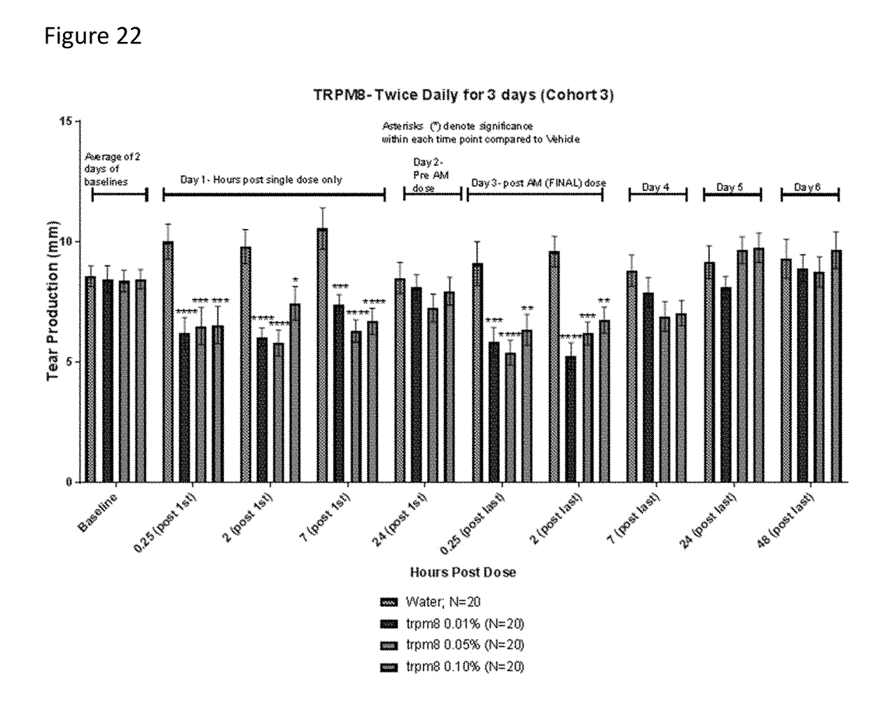

For Cohort 2, baselines were obtained on Days 1, 2 and 5. Tear production was measured for each mouse for 15 seconds per each eye. Mice received doses of either 0.1% TRPM8, 0.05% TRPM8, 0.01% TRPM8, or water once on Days 9-11. Mice had tear production measured at time points: 15 mins, 2 hours, 7 hours and 24 hours post 1st dose and last dose and 48 hours and 72 hours post last dose. FIG. 22 shows the results for Cohort 2. Table 15, below, shows the results of two-way ANOVA compared to respective baseline for Cohort 2.

TABLE 15
Cohort 2 OU

| Trpm8 0. 10% (N = 20) | | Trpm8 0.01% (N = 20) | |
|---|---|---|---|
| Baseline vs. 0.25 (post 1st) | **** | Baseline vs. 0.25 (post 1st) | **** |
| Baseline vs. 2 (post 1st) | ** | Baseline vs. 2 (post 1st) | ns |
| Baseline vs. 7 (post 1st) | ns | Baseline vs. 7 (post 1st) | * |
| Baseline vs. 24 (post 1st) | ns | Baseline vs. 24 (post 1st) | ns |
| Baseline vs. 0.25 (post last) | ** | Baseline vs. 0.25 (post last) | ns |
| Baseline vs. 2 (post last) | ** | Baseline vs. 2 (post last) | ns |
| Baseline vs. 7 (post last) | ns | Baseline vs. 7 (post last) | ns |
| Baseline vs. 24 (post last) | ns | Baseline vs. 24 (post last) | ns |
| Baseline vs. 48 (post last) | ns | Baseline vs. 48 (post last) | ns |
| Baseline vs. 72 (post last | ns | Baseline vs. 72 (post last | ns |
| dose) | | dose) | |
| Trpm8 0.05% (N = 20) | | Water (N = 20) | |
| Baseline vs. 0.25 (post 1st) | **** | Baseline vs. 0.25 (post 1st) | ns |
| Baseline vs. 2 (post 1st) | ns | Baseline vs. 2 (post 1st) | ns |
| Baseline vs. 7 (post 1st) | ns | Baseline vs. 7 (post 1st) | * |
| Baseline vs. 24 (post 1st) | ns | Baseline vs. 24 (post 1st) | ns |
| Baseline vs. 0.25 (post last) | ns | Baseline vs. 0.25 (post last) | ** |
| Baseline vs. 2 (post last) | ns | Baseline vs. 2 (post last) | ** |
| Baseline vs. 7 (post last) | ns | Baseline vs. 7 (post last) | ns |
| Baseline vs. 24 (post last) | ns | Baseline vs. 24 (post last) | ns |
| Baseline vs. 48 (post last) | ns | Baseline vs. 48 (post last) | ns |
| Baseline vs. 72 (post last | ns | Baseline vs. 72 (post last | ns |
| dose) | | dose) | |

### Cohort 3

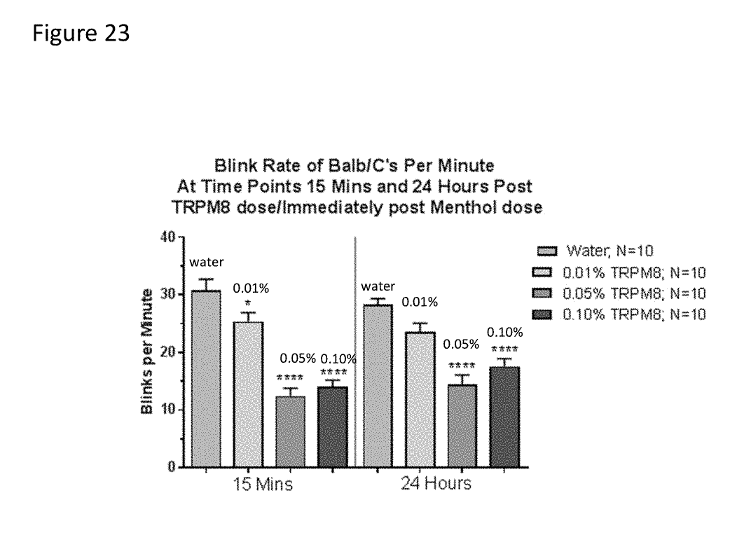

For Cohort 3, baselines were obtained on Days 36, 29 and 40. Tear production was measured for each mouse for 15 seconds per each eye. Mice received doses of either 0.1% TRPM8, 0.05% TRPM8, 0.01% TRPM8, or water twice on Days 43-44, once on Day 45. Mice had tear production measured at time points: 15 mins, 2 hours, 7 hours and 24 hours post 1st dose and last dose and 48 hours post last dose. FIG. 23 shows the results for Cohort 3. Table 16, below, shows the results of two-way ANOVA compared to respective baseline for Cohort 3.

### Blink Rate

The blink rate of Balb/C mice per minute at time points 15 minutes and 24 hours post TRPM8 dose/immediately post menthol dose was examined The results are shown in FIG. 23. Changes in observed blink rates from 15 minutes and 24 hours post treatment illustrate the effects of both Menthol and TRPM8 antagonist in comparison to base.

TABLE 16
Cohort 3 OU

| Trpm8 0.10% (N = 20) | | Trpm8 0.01% (N = 20) | |
|---|---|---|---|
| Baseline vs. 0.25 (post 1st) | * | Baseline vs. 0.25 (post 1st) | ** |
| Baseline vs. 2 (post 1st) | ns | Baseline vs. 2 (post 1st) | *** |
| Baseline vs. 7 (post 1st) | * | Baseline vs. 7 (post 1st) | ns |
| Baseline vs. 24 (post 1st) | ns | Baseline vs. 24 (post 1st) | ns |
| Baseline vs. 0.25 (post last) | ** | Baseline vs. 0.25 (post last) | *** |
| Baseline vs. 2 (post last) | * | Baseline vs. 2 (post last) | **** |
| Baseline vs. 7 (post last) | ns | Baseline vs. 7 (post last) | ns |
| Baseline vs. 24 (post last) | ns | Baseline vs. 24 (post last) | ns |
| Baseline vs. 48 (post last) | ns | Baseline vs. 48 (post last) | ns |
| Trpm8 0.05% (N = 20) | | Water (N = 20) | |
| Baseline vs. 0.25 (post 1st) | * | Baseline vs. 0.25 (post 1st) | ns |
| Baseline vs. 2 (post 1st) | *** | Baseline vs. 2 (post 1st) | ns |
| Baseline vs. 7 (post 1st) | ** | Baseline vs. 7 (post 1st) | ** |
| Baseline vs. 24 (post 1st) | ns | Baseline vs. 24 (post 1st) | ns |
| Baseline vs. 0.25 (post last) | **** | Baseline vs. 0.25 (post last) | ns |
| Baseline vs. 2 (post last) | ** | Baseline vs. 2 (post last) | ns |
| Baseline vs. 7 (post last) | ns | Baseline vs. 7 (post last) | ns |
| Baseline vs. 24 (post last) | ns | Baseline vs. 24 (post last) | ns |
| Baseline vs. 48 (post last) | ns | Baseline vs. 48 (post last) | ns |

### References

NOCICEPTORS, P. (2011). Cold thermoreceptors, unexpected players in tear production and ocular dryness sensations. Investigative ophthalmology & visual science, 52(6), 3888. Hirata H Meng I D. Cold-sensitive corneal afferents respond to a variety of ocular stimuli central to tear production: implications for dry eye disease. Invest Ophthalmol Vis Sci. 2010; 51:3969-3976.
Robbins, A., Kurose, M., Winterson, B. J., & Meng, I. D. (2012). Menthol activation of corneal cool cells induces TRPM8-mediated lacrimation but not nociceptive responses in rodents. Investigative ophthalmology & visual science, 53(11), 7034.
Kurose, M., & Meng, I. D. (2013). Dry eye modifies the thermal and menthol responses in rat corneal primary afferent cool cells. Journal of neurophysiology, 110(2), 495-504.
McKemy, David D., Werner M. Neuhausser, and David Julius. “Identification of a cold receptor reveals a general role for TRP channels in thermosensation.”Nature 416.6876 (2002): 52-58.

Galeotti, N., Mannelli, L. D. C., Mazzanti, G., Bartolini, A., & Ghelardini, C. (2002). Menthol: a natural analgesic compound. Neuroscience letters, 322(3), 145-148.

Bourcier, T., Acosta, M. C., Borderie, V., Borrás, F., Gallar, J., Bury, T., . . . & Belmonte, C. (2005). Decreased corneal sensitivity in patients with dry eye. Investigative ophthalmology & visual science, 46(7), 2341-2345.

1. A method of identifying a subject as susceptible to treatment for an ocular disease or disorder, comprising:
administering a transient receptor potential melastatin 8 (TRPM8) agonist to the eye of the subject;
detecting the effect of the TRPM8 agonist on the symptoms of the ocular disease or disorder; and
identifying the subject as susceptible to treatment for the ocular disease or disorder based on the results of the detecting step.

2. A method of measuring the severity of the symptoms of an ocular disease or disorder in a subject, comprising:
administering a transient receptor potential melastatin 8 (TRPM8) agonist to the eye of the subject;
detecting the effect of the TRPM8 agonist on the symptoms of the ocular disease or disorder; and
assessing the effect of the TRPM8 agonist on the severity of the symptoms of the ocular disease or disorder using one or more dry eye questionnaires.

3. (canceled)

4. The method of claim 1, further comprising treating the subject for the ocular disease or disorder.

5. The method of claim 1, wherein the TRPM8 agonist is menthol.

6. The method of claim 5, wherein the menthol is administered at a concentration of about 0.0001% to about 0.1% w/v.

7. The method of claim 5, wherein the menthol is administered at a concentration of about 0.000050% to about 0.000075% w/v.

8. The method of claim 1, wherein the effect of the TRPM8 agonist on the symptoms of the ocular disease or disorder is determined using one or more dry eye questionnaires.

9. (canceled)

10. The method of claim 1, further comprising measuring the duration of the effect of the TRPM8 agonist on the symptoms of the ocular disease or disorder.

11. The method of claim 10, wherein the duration of the effect of the TRPM8 agonist on the symptoms of the ocular disease or disorder is used to identify the subject as susceptible to treatment for the ocular disease or disorder.

12. (canceled)

13. The method of claim 1, wherein the subject has an ocular disease or disorder.

14. The method of claim 13, wherein the subject has had the ocular disease or disorder for less than 10 years.

15. The method of claim 13, wherein the subject has had the ocular disease or disorder for greater than 10 years.

16. (canceled)

17. A method of screening an agent for the treatment of an ocular disease or disorder, comprising:
administering a TRPM8 agonist to the eye of a subject having an ocular disease or disorder;
administering the agent to the eye of the subject at a concentration that alleviates symptoms of the ocular disease or disorder; and
determining an effect of the agent on the symptoms of the ocular disease or disorder.

18. A method of screening an agent for the treatment of an ocular disease or disorder, comprising:
administering the agent to the eye of a subject;
administering a TRPM8 agonist to the eye of the subject to elicit symptoms of the ocular disease or disorder; and
determining an effect of the TRPM8 agonist on the symptoms of the ocular disease or disorder.

19. The method of claim 18, wherein the subject has an ocular disease or disorder.

20. The method of claim 17, wherein the agent is a transient receptor potential melastatin 8 (TRPM8) antagonist.

21. The method of claim 17, wherein the TRPM8 agonist is menthol.

22. The method of claim 21, wherein the menthol is administered at a concentration of about 0.0001% to about 0.1% w/v.

23. (canceled)

24. (canceled)

25. (canceled)

26. (canceled)

27. The method of claim 1, wherein the ocular disease or disorder is ocular discomfort.

28. The method of claim 1, wherein the ocular disease or disorder is dry eye disease or dry eye discomfort.

29. (canceled)

30. (canceled)

31. (canceled)

32. (canceled)
